(19) United States
(12) Patent Application Publication
(10) Pub. No.: US 2024/0058331 A1
(43) Pub. Date: Feb. 22, 2024

(54) COMBINATION THERAPIES FOR TREATMENT OF CIRRHOSIS WITH PORTAL HYPERTENSION

(71) Applicant: AstraZeneca AB, Södertälje (SE)

(72) Inventors: Philip AMBERY, Södertälje (SE); Peter GREASLEY, Södertälje (SE)

(21) Appl. No.: 18/448,559

(22) Filed: Aug. 11, 2023

Related U.S. Application Data
(60) Provisional application No. 63/371,228, filed on Aug. 12, 2022.

(51) Int. Cl. A61K 31/497; A61K 31/7034; A61K 9/00; A61P 1/16

(52) U.S. Cl. CPC A61K 31/497; A61K 31/7034; A61K 9/0053; A61P 1/16

(57) ABSTRACT

A method of treating liver cirrhosis in a subject in need thereof. The method includes administering to the subject a composition. The composition includes a therapeutically effective amount of zibotentan and dapagliflozin.

### FIELD

The present disclosure provides methods and compositions for treating liver cirrhosis in a subject in need thereof, and in particular, to compositions comprising a therapeutically effective amount of zibotentan and dapagliflozin, and methods of administering the compositions.

### BACKGROUND

Cirrhosis is the end-stage result of chronic liver injury or chronic liver disease and can be characterized by advanced fibrosis. Common causes of cirrhosis include viral hepatitis (hepatitis B and C), alcoholic liver disease, and non-alcoholic steatohepatitis (NASH), while less common causes of cirrhosis include autoimmune hepatitis, primary sclerosing cholangitis, primary biliary cholangitis, haemochromatosis, biliary atresia, alpha-1-antitrypsin deficiency and cystic fibrosis related liver cirrhosis, and Wilson's disease (Romanelli et al., 2016). Cirrhosis may be compensated in which no clinical complications that affect outcome (i.e., decompensation events such as variceal haemorrhage, ascites, hepatorenal syndrome or acute kidney injury, hepatic encephalopathy, hepatocellular cancer) have occurred, or decompensated, in which patients have had at least one clinical complication. In decompensated patients, the liver has reduced ability to perform vital metabolic, synthetic and storage functions (Tsochatzis et al., 2014). Cirrhosis can be a serious condition which leads to significant morbidity, resource intensive complications, hepatocellular carcinoma, and, in the absence of liver transplantation, death. The Global Burden of Disease Study in 2017 reported over 1.32 million cirrhosis-related deaths globally, which was approximately 2.4% of all deaths worldwide (GBD 2017 Cirrhosis Collaborators, 2020).

Portal hypertension is a common feature of chronic liver disease. The primary cause of portal hypertension in cirrhosis is an increase in intrahepatic vascular resistance due to structural changes associated with fibrosis and increased vascular tone in the hepatic microcirculation (Iwakiri, 2014). As portal hypertension develops, arterial vasodilation occurs, which results in activation of RAAS and sympathetic nervous system and result in increased blood flow to the portal circulation (Iwakiri, 2014). Eventually, a hyperdynamic circulatory syndrome can develop, leading to a further increase in portal hypertension, formation of collateral vessels and variceal hemorrhage, hepatic encephalopathy and/or ascites (Poordad, 2015).

Cirrhosis patients with features of clinically significant portal hypertension have significant risk of decompensation and death but have limited pharmacological treatment options. Standard treatment for patients with chronic liver disease centers upon treatment of the underlying cause of liver disease, e.g., abstinence from alcohol, anti-viral agents for viral hepatitis, and treatment of underlying metabolic disease in NASH. Although some viruses can now be eradicated, given the continued emergence of fatty liver diseases, disease modifying therapies are generally expected to have marginal impact on the outcome in patients with cirrhosis with portal hypertension (Gunarathne et al., 2020). Other treatments may include the use of non-selective beta blockers, diuretics, and vasoactive drugs (Gunarathne et al., 2020). In patients developing variceal haemorrhage, intervention with transjugular intrahepatic portosystemic shunt (TIPS) may be used to reduce portal hypertension (García-Pagán et al., 2020). However, none of the above guidelines and intervention can be used to efficiently treat cirrhosis patients with features of portal hypertension.

### SUMMARY

The present disclosure provides a method of treating liver cirrhosis in a subject in need thereof. In some embodiments, the method comprises administering to the subject a composition, and the composition comprises a therapeutically effective amount of zibotentan and dapagliflozin. In some embodiments, the subject has portal hypertension.

In some embodiments, the subject has a hepatic venous pressure gradient (HVPG) greater than 5 mmHg. In some embodiments, the subject has an HVPG greater than 7.5 mmHg. In some embodiments, the subject has an HVPG greater than 10 mmHg. In some embodiments, the subject has an HVPG greater than 12 mmHg

In some embodiments, the subject has at least one of oesophageal varices, splenomegaly, variceal hemorrhages, recurrent variceal hemorrhages, ascites, refractory ascites, hepatic encephalopathy, recurrent hepatic encephalopathy, lower extremity swelling, coagulopathy, pulmonary complications, and portosystemic shunts.

In some embodiments, the subject has a Child-Pugh score that equals to or is greater than 5. In some embodiments, the subject has a Child-Pugh score that equals to or is greater than 7. In some embodiments, the subject has a Child-Pugh score that equals to or is greater than 10. In some embodiments, Child-Pugh score is not measured in a subject.

In some embodiments, the subject has a model for end-stage liver disease (MELD) score that equals to or is greater than 5. In some embodiments, the subject has an MELD score that equals to or is greater than 10. In some embodiments, the subject has an MELD score that equals to or is greater than 20. In some embodiments, the subject has an MELD score that equals to or is greater than 25. In some embodiments, MELD score is not measured in a subject.

In some embodiments, the subject has no decompensation events. In some embodiments, the subject has decompensation events.

In some embodiments, the composition comprises about 0.1 mg to about 240 mg zibotentan. In some embodiments, the composition comprises about 0.2 mg to about 50 mg zibotentan. In some embodiments, the composition comprises about 0.5 mg to about 20 mg zibotentan. In some embodiments, the composition comprises about 1 mg to about 10 mg zibotentan.

In some embodiments, the composition comprises about 1 mg zibotentan. In some embodiments, the composition comprises about 2.5 mg zibotentan. In some embodiments, the composition comprises about 5 mg zibotentan.

In some embodiments, the composition comprises about 0.1 mg to about 100 mg dapagliflozin. In some embodiments, the composition comprises about 0.25 mg to about 50 mg dapagliflozin. In some embodiments, the composition comprises about 1 mg to about 10 mg dapagliflozin.

In some embodiments, the composition comprises about 2.5 mg dapagliflozin. In some embodiments, the composition comprises about 5 mg dapagliflozin. In some embodiments, the composition comprises about 10 mg dapagliflozin.

In some embodiments, the composition comprises about 1 mg zibotentan and about 2.5 mg dapagliflozin. In some embodiments, the composition comprises about 1 mg zibotentan and about 5 mg dapagliflozin. In some embodiments, the composition comprises about 1 mg zibotentan and about 10 mg dapagliflozin.

In some embodiments, the composition comprises about 2.5 mg zibotentan and about 2.5 mg dapagliflozin. In some embodiments, the composition comprises about 2.5 mg zibotentan and about 5 mg dapagliflozin. In some embodiments, the composition comprises about 2.5 mg zibotentan and about 10 mg dapagliflozin.

In some embodiments, the composition comprises about 5 mg zibotentan and about 5 mg dapagliflozin. In some embodiments, the composition comprises about 5 mg zibotentan and about 10 mg dapagliflozin.

In some embodiments, the composition is in an oral dosage form comprising zibotentan and dapagliflozin. In some embodiments, the oral dosage form is a capsule or tablet.

In some embodiments, the composition is administered to the subject once to three times daily. In some embodiments, the composition is administered to the subject once daily.

In some embodiments, the composition is administered to the subject at least once to three times daily for at least 3 weeks. In some embodiments, the composition is administered to the subject at least once to three times daily for at least 6 weeks. In some embodiments, the composition is administered to the subject at least once to three times daily for at least 12 weeks. In some embodiments, the composition is administered to the subject at least once to three times daily for at least 16 weeks. In some embodiments, the composition is administered to the subject at least one to three times daily for an extended period of time greater than 16 weeks (32 weeks, 64 weeks, 1 year, 5 years, or longer) depending on the requirements of the subject(s).

In some embodiments, the subject has a hepatic venous pressure gradient (HVPG) response and the HVPG is decreased at least 10-40% after administration of the composition to the subject relative to a baseline HVPG of the subject. In some embodiments, the HVPG is decreased at least 10% after administration of the composition to the subject relative to a baseline HVPG of the subject. In some embodiments, the HVPG of the subject is decreased at least 15% after administration of the composition to the subject. In some embodiments, the HVPG of the subject is decreased at least 20% after administration of the composition to the subject. In some embodiments, the HVPG of the subject is decreased at least 25% after administration of the composition to the subject. In some embodiments, the HVPG of the subject is decreased at least 30% after administration of the composition to the subject. In some embodiments, the HVPG of the subject is decreased at least 35% after administration of the composition to the subject. In some embodiments, the HVPG of the subject is decreased at least 40% after administration of the composition to the subject.

In some embodiments, the subject has a reduction of hepatic venous pressure gradient (HVPG) to less than or equal to 12 mmHg after administration of the composition to the subject relative to a baseline HVPG of the subject. In some embodiments, the HVPG of the subject is less than or equal to 10 mmHg after administration of the composition to the subject. In some embodiments, the HVPG of the subject is less than or equal to 8 mmHg after administration of the composition to the subject. In some embodiments, the HVPG of the subject is reduced down to less than or equal to 5 mmHg after administration of the composition to the subject.

In some embodiments, the subject's HVPG is reduced at least 1.0 mmHg after administration of the composition to the subject. In some embodiments, the subject's HVPG is reduced at least 1.5 mmHg after administration of the composition to the subject. In some embodiments, the subject's HVPG is reduced at least 2.0 mmHg after administration of the composition to the subject. In some embodiments, the subject's HVPG is reduced at least 2.5 mmHg after administration of the composition to the subject.

In some embodiments, the subject has one or more of the following outcomes after administration of the composition to the subject: (i) at least 5% decrease in body weight; (ii) at least 5% reduction in body water volume; (iii) at least 5% reduction in body fat mass; (iv) systolic blood pressure is reduced to less than 130; (v) diastolic blood pressure is reduced to less than 90; and (vi) at least 5% reduction in accumulated additional loop-diuretic equivalents use.

In some embodiments, the subject has one or more of the following outcomes after administration of the composition to the subject: (i) at least 10% decrease in body weight; (ii) at least 10% reduction in body water volume; (iii) at least 10% reduction in body fat mass; (iv) systolic blood pressure is reduced to less than 120; (v) diastolic blood pressure is reduced to less than 80; and (vi) at least 10% reduction in accumulated additional loop-diuretic equivalents use.

In some embodiments, the subject has one or more of the following outcomes after administration of the composition to the subject: (i) at least 15% decrease in body weight; (ii) at least 15% reduction in body water volume; (iii) at least 15% reduction in body fat mass; (iv) systolic blood pressure is reduced to less than 110; (v) diastolic blood pressure is reduced to less than 70; and (vi) at least 15% reduction in accumulated additional loop-diuretic equivalents use.

In some embodiments, the liver cirrhosis results from viral hepatitis (e.g., Hepatitis B or Hepatitis C), alcoholic liver disease, hepatic steatosis, non-alcoholic fatty liver disease (NAFLD), non-alcoholic steatohepatitis (NASH), or combinations thereof.

In some embodiments, the liver cirrhosis results from autoimmune hepatitis, primary sclerosing cholangitis, primary biliary cholangitis, haemochromatosis, Wilson's disease, biliary atresia, alpha-1-antitrypsin deficiency and cystic fibrosis related liver cirrhosis, or combinations thereof.

In some embodiments, the subject is a human subject. In some embodiments, the human subject is 18 years to 80 years of age.

In certain aspects, the present disclosure relates to a kit. In some embodiments, the kit comprising: (a) an oral dosage form comprising about 0.25 mg to about 20 mg zibotentan, and a pharmaceutically acceptable excipient; and (b) an oral dosage form comprising about 0.5 mg to about 40 mg dapagliflozin, and a pharmaceutically acceptable excipient.

In certain aspects, the present disclosure relates to a kit. In some embodiments, the kit comprising: (a) an oral dosage form comprising about 2.5 mg zibotentan, and a pharmaceutically acceptable excipient; and (b) an oral dosage form comprising about 5 mg dapagliflozin, and a pharmaceutically acceptable excipient.

In certain aspects, the present disclosure relates to a method of reducing the risk of hospitalization, trans jugular intrahepatic portosystemic shunt, and/or transplantation in a subject in need thereof. In some embodiments, the method comprises administering to the subject a composition comprising a therapeutically effective amount of zibotentan and dapagliflozin. In certain embodiments, reducing the risk of hospitalization may mean first or recurrent hospitalizations or total number of hospitalizations. In certain embodiments, reducing the risk of hospitalization may mean reducing the risk of hospitalization for decompensation events.

In certain aspects, the present disclosure relates to a method for reducing fluid overload associated with liver cirrhosis in a subject in need thereof. In some embodiments, the method comprises administering to the subject a composition comprising a therapeutically effective amount of zibotentan and dapagliflozin.

In certain aspects, the present disclosure relates to a method for reducing the risk of all-cause mortality associated with liver cirrhosis in a subject in need thereof. In some embodiments, the method comprises administering to the subject a composition comprising a therapeutically effective amount of zibotentan and dapagliflozin.

In certain aspects, the present disclosure relates to a method of reducing hepatic portal venous pressure in a subject in need thereof. In some embodiments, the method comprises administering to the subject a composition comprising a therapeutically effective amount of zibotentan and dapagliflozin.

In certain aspects, the present disclosure relates to a method of reducing Child Pugh score and/or reducing progression of Child-Pugh score in a subject in need thereof. In some embodiments, the method comprises administering to the subject a composition comprising a therapeutically effective amount of zibotentan and dapagliflozin.

In certain aspects, the present disclosure relates to a method of reducing MELD score and/or reducing progression of MELD score in a subject in need thereof. In some embodiments, the method comprises administering to the subject a composition comprising a therapeutically effective amount of zibotentan and dapagliflozin.

In certain aspects, the present disclosure relates to a method of treating liver cirrhosis or preventing and/or delaying progression of liver cirrhosis, in a subject in need thereof. In some embodiments, the method comprises administering to the subject a composition comprising a therapeutically effective amount of zibotentan and dapagliflozin.

In certain aspects, the present disclosure relates to a method of treating liver cirrhosis with portal hypertension or preventing and/or delaying progression of liver cirrhosis with portal hypertension, in a subject in need thereof. In some embodiments, the method comprises administering to the subject a composition comprising a therapeutically effective amount of zibotentan and dapagliflozin.

In certain aspects, the present disclosure relates to a method of treating liver cirrhosis with clinically significant portal hypertension or preventing and/or delaying progression of liver cirrhosis with clinically significant portal hypertension, in a subject in need thereof. In some embodiments, the method comprises administering to the subject a composition comprising a therapeutically effective amount of zibotentan and dapagliflozin.

In certain aspects, the present disclosure relates to a method of reducing the risk of cirrhosis decompensation or preventing and/or delaying progression to cirrhosis decompensation events, in a subject in need thereof. In some embodiments, the method comprises administering to the subject a composition comprising a therapeutically effective amount of zibotentan and dapagliflozin.

In certain aspects, the present disclosure relates to a method of reducing the risk of liver-related decompensation events or death and/or preventing and/or delaying progression to liver-related decompensation events or death, in a subject in need thereof. In some embodiments, the method comprises administering to the subject a composition comprising a therapeutically effective amount of zibotentan and dapagliflozin.

In certain aspects, the present disclosure relates to a method of reducing the risk of variceal haemorrhage and/or death or a method of preventing and/or delaying progression to variceal haemorrhage and/or death, in a subject in need thereof. In some embodiments, the method comprises administering to the subject a composition comprising a therapeutically effective amount of zibotentan and dapagliflozin.

In certain aspects, the present disclosure relates to a method of treating ascites and/or preventing or reducing the formation of ascites and/or delaying progression of ascites and/or delaying progression to complications of ascites, in a subject in need thereof. In some embodiments, the method comprises administering to the subject a composition comprising a therapeutically effective amount of zibotentan and dapagliflozin.

### BRIEF DESCRIPTION OF THE DRAWINGS

The following drawings form part of the present specification and are included to further demonstrate exemplary embodiments of certain aspects of the present disclosure.

FIG. 1 shows study design of Example 1. Dapa=dapagliflozin; HVPG=hepatic venous pressure gradient; N=number of participants; Zibo=zibotentan.

### DETAILED DESCRIPTION

Unless otherwise defined herein, scientific, and technical terms used in the present disclosure shall have the meanings that are commonly understood by one of ordinary skill in the art.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% or higher variability (either “greater than” or “less than” the indicated value), depending on the situation. In embodiments, one of skill in the art will understand the level of variability indicated by the term “about,” due to the context in which it is used herein. It should also be understood that use of the term “about” also includes the specifically recited value.

Ranges provided herein, of any type, include all values within a particular range described and values about an endpoint for a particular range. As used herein, “between” is a range inclusive of the ends of the range. For example, a number between x and y explicitly includes the numbers x and y, and any numbers that fall within x and y.

The term “dose” means a specified quantity of a compound or pharmaceutical agent provided in a single administration, or in a specified time period. In certain embodiments, a dose may be administered in two or more boluses, tablets, or injections. For example, in certain embodiments, where subcutaneous administration is desired, the desired dose may require a volume not easily accommodated by a single injection. In such embodiments, two or more injections may be used to achieve the desired dose. In certain embodiments, a dose may be administered in two or more injections to minimize injection site reaction in an individual. In other embodiments, the compound or pharmaceutical agent is administered by infusion over an extended period of time or continuously. Doses may be stated as the amount of pharmaceutical agent per hour, day, week or month.

The term “efficacy” means the ability to produce a desired effect.

The term “treating” or “treatment” refers to administering a compound or pharmaceutical composition to an animal in order to effect an alteration or improvement of a disease, disorder, or condition in the animal.

The term “liver cirrhosis” refers to a late-stage result of liver disease in which the liver is markedly scarred and damaged. Scar tissue replaces healthy liver tissue and prevents the liver from working normally. As cirrhosis gets worse, the liver begins to fail.

The term “subject” is meant any subject, particularly a mammalian subject, in need of treatment with a combination of zibotentan and dapagliflozin, or treatment with dapagliflozin only. Mammalian subjects include human or non-human animal. In some embodiments, the term “subject” refers to a human subject. In some embodiments, the term “subject” refers to a female subject. In some embodiments, the term “subject” refers to a male subject. In some embodiments, the human subject is 12 years of age or older, 14 years of age or older, 4-17 years of age or older, 18 years of age or older. In some embodiments, the human subject is 18 years to 80 years of age.

Non-human animal includes, but are not limited to, pigs, dogs, cats, guinea pigs, rabbits, rats, mice, horses, cattle, bears, cows, apes, monkeys, orangutans, and chimpanzees, and so on. As used herein, a “subject in need thereof” refers to the subject for whom it is desirable to treat, e.g., a subject having cirrhosis with portal hypertension.

Zibotentan is a compound of the following Formula 1:

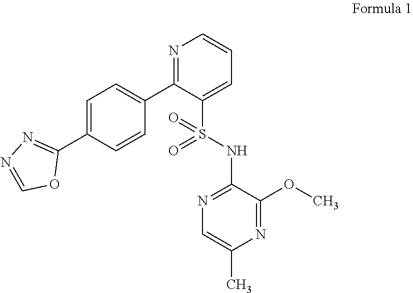 [SMILES: COc1nc(C)cnc1NS(=O)(=O)c1cccnc1-c1ccc(-c2nnco2)cc1]

Zibotentan, or ZD4054, has been disclosed in WO1996040681 and is a selective endothelin (ET_{A}) receptor antagonist with a high level of selectivity over ET_{B} (ET_{A} IC₅₀=13 nM, no effect at the ET_{B} up to 50 mM) (Morris et al., “Specific inhibition of the endothelin A receptor with ZD4054: clinical and pre-clinical evidence,” Br J Cancer, 2005, 92(12):2148-2152), previously developed as an anti-cancer drug candidate. While not being bound by any particular mechanism of action, zibotentan may be an antagonist of endothelin (ET-1) mediated vasoconstriction in the portal vasculature and intrahepatic stellate cells, which may lead to a reduction in portal venous pressure in established portal hypertension. Studies of beta blockers in cirrhosis have established that reduced portal hypertension improves outcomes and can reduce the risk of decompensation events and perhaps death. Non-selective beta-blockers reduce portal hypertension mainly by reducing blood flow in the portal vein and venous collaterals. Zibotentan may provide further benefit through its effect on hepatic vascular resistance (reduced) and improve endothelial function, and may also exhibit, in the longer term, anti-fibrotic effects in the liver, which would be expected to improve overall liver function. However, as a chronic monotherapy, the benefit:risk profile for zibotentan in cirrhosis may be unfavorable given the potential for ET-1 antagonism to cause fluid retention in some participants.

Dapagliflozin is a compound of Formula 2:

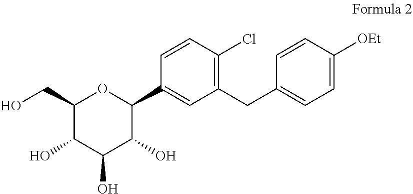 [SMILES: CCOc1ccc(Cc2cc([C@@H]3O[C@H](CO)[C@@H](O)[C@H](O)[C@H]3O)ccc2Cl)cc1]

Dapagliflozin is a potent, selective, and orally active inhibitor of sodium-glucose cotransporter 2 (SGLT2), with IC₅₀=3.75 nM. Dapagliflozin was originally developed to improve glycemic control in patients with diabetes mellitus and provides cardiorenal benefits in patients with and without type 2 diabetes mellitus (T2DM). Dapagliflozin has also been used to treat certain kinds of heart failure, and chronic kidney disease. SGLT2 inhibition blocks glucose and sodium reabsorption in the proximal tubule of the kidney leading to an osmotic diuresis without subsequent hyponatraemia or hyperkalaemia, while counteracting the effects of renin-angiotensin-aldosterone system activation.

In some embodiments, the at least one compound chosen from compounds of Formula 2. In some embodiments, the at least one compound is in the form of a pharmaceutically acceptable solvate, mixed solvate, or complex. In some embodiments, the at least one compound is in the form of a non-crystalline solid (e.g., amorphous form). In some embodiments, the at least one compound is in the form of a crystalline solid.

In some embodiments, the at least one compound is in the form of a (S)-propylene glycol ((S)-PG) solvate which has the structure shown below:

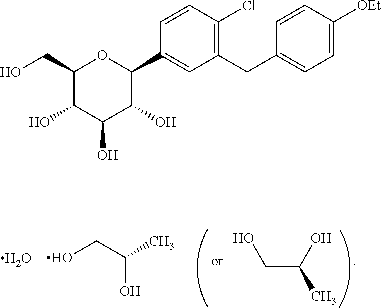

In some embodiments, the at least one compound is in the form of a crystalline S-PG solvate. Methods for preparing a (S)-PG solvate of dapagliflozin, including a crystalline S-PG solvate, are provided in U.S. Pat. No. 7,919,598.

In some embodiments, the method comprises administering to a subject in need thereof a pharmaceutical composition comprising a therapeutically effective amount of at least one compound chosen from compounds of Formula 2 and prodrugs thereof alone or in combination with at least one other therapeutic agent.

The mechanisms of action of zibotentan and dapagliflozin are different. While not being bound by any particular theory, in some embodiments, zibotentan can block endothelin 1 driven changes in intrahepatic resistance (including elements of portal vein and sinusoidal vasoconstriction), decrease vascular stiffness, improve endothelial function, reduce hepatic inflammation and, in the long term, potentially reduce fibrosis. Separately, in some embodiments, dapagliflozin can have beneficial effects on metabolic dysfunction, reduce hepatocyte injury and sodium and water overload potentially normalizing blood volume, while avoiding hyponatraemia and hyperkalaemia. A reduced need for diuretics may also reduce the risk for potential acute kidney injury or other complications such as encephalopathy.

In some embodiments, the subject has portal hypertension. The term “portal hypertension” refers to abnormally increased portal venous pressure—blood pressure in the portal vein and its branches that drain from most of the intestine to the liver. In some embodiments, portal hypertension is defined as a hepatic venous pressure gradient (HVPG) greater than 5 mmHg. In some embodiments, it is defined as clinically significant when the HVPG is greater than 10 mmHg. In subjects with liver cirrhosis, HVPG represents the portal venous pressure. In some embodiments, the subject has a HVPG greater than 4 mmHg, 5 mmHg, 6 mmHg, 7 mmHg, 8 mmHg, 9 mmHg, 10 mmHg, 11 mmHg or 12 mmHg. In some embodiments, the subject has a HVPG greater than 5 mmHg. In some embodiments, the subject has an HVPG greater than 7.5 mmHg. In some embodiments, the subject has an HVPG greater than 10 mmHg. In some embodiments, the subject has an HVPG greater than 12 mmHg.

In some embodiments, the HVPG are measured by methods known to the skilled artisan, either directly or indirectly. In some embodiments, the HVPG is measured directly, e.g., by direct catherization of the portal vein, e.g., Myers J. D., Taylor W. J., “An estimation of portal venous pressure by occlusive catheterization of a hepatic venule,” J Clin Invest, 30 (1951), pp. 662-663. In some embodiments, the HVPG is measured indirectly, e.g., a balloon catheter is used to wedge (occlude) the hepatic vein. See, e.g., Groszmann R. J., Glickman M. C., Blei A. T., Storer E., Conn H. O., “Wedged and free hepatic venous pressure measured with a balloon catheter,” Gastroenterology, 76 (1979), pp. 253-258.

In some embodiments, HVPG is a clinical measurement of the pressure gradient between the wedged hepatic venous pressure (WHVP) and the free hepatic venous pressures (FHPV), and thus is an estimate of the pressure gradient between the portal vein and the inferior vena cava. An HVPG of >5 mmHg defines portal hypertension, and if the measurement exceeds 10 mmHg it is called clinically significant portal hypertension. At levels above 12 mm Hg, the risk for variceal haemorrhage may further increase.

In some embodiments, when blood flow in a hepatic vein is stopped by an inflated balloon, the proximal static column of blood transmits the pressure from the preceding communicated vascular territory (hepatic sinusoids) to the catheter. Thus, WHVP reflects hepatic sinusoidal pressure and not the portal pressure itself. In the normal liver, due to pressure equilibration through interconnected sinusoids, wedged pressure is slightly lower than portal pressure, though this difference is clinically insignificant. In liver cirrhosis, the static column created by balloon inflation cannot be decompressed at the sinusoidal level due to disruption of the normal intersinusoidal communications; therefore, HVPG (WHVP-FHVP) gives an accurate estimation of portal pressure in cirrhosis.

In some embodiments, the HVPG is not measured either directly or indirectly, but is determined by observation of features consistent with portal hypertension in the subject. In some embodiments, the subject has clinically significant portal hypertension (CSPH). In some embodiments, the term “features of portal hypertension” includes those signs and symptoms in a subject with compensated cirrhosis associated with CSPH, e.g., oesophageal varices, portosystemic shunts, splenomegaly, and low platelet count or the subject has had a decompensation event including variceal hemorrhages, recurrent variceal hemorrhages, ascites, refractory ascites, hepatic encephalopathy, recurrent hepatic encephalopathy.

In some embodiments, the subject has decompensation events. In some embodiments, “decompensation events” can include ascites, hepatic encephalopathy, hepatorenal syndrome or acute kidney injury or variceal haemorrhage. In some embodiments, the subject manifests (including onset, worsening and/or complications of) one or more decompensation events, e.g., oesophageal varices, variceal hemorrhages, recurrent variceal hemorrhages, ascites, refractory ascites, hepatic encephalopathy, recurrent hepatic encephalopathy, jaundice, or pulmonary complications. In some embodiments, the subject has no decompensation events. In some embodiments, “Child-Pugh score” is used to assess the prognosis of chronic liver disease, mainly cirrhosis. Although “Child-Pugh score” was originally used to predict mortality during surgery, it is now used to determine the prognosis, as well as the required strength of treatment and the necessity of liver transplantation. See, e.g., Andrea Tsoris; Clinton A. Marlar, “Use of the Child Pugh Score in Liver Disease,” [Updated 2022 Mar. 18]. In: StatPearls [Internet]. Treasure Island (FL): StatPearls Publishing; (2022), Available from: https://www.ncbi.nlm.nih.gov/books/NBK542308/, accessed Jun. 29, 2022. Chronic liver disease can be classified into Child-Pugh class A to C, where class A has a Child-Pugh score of 5-6, class B has a Child-Pugh score of 7-9, and class C has a Child-Pugh score of 10-15. The higher the Child-Pugh score, the higher the mortality rate.

In some embodiments, the subject has a Child-Pugh score that equals to or is greater than 5, equals to or is greater than 6, equals to or is greater than 7, equals to or is greater than 8, equals to or is greater than 9, or equals to or is greater than 10. In some embodiments, the subject has a Child-Pugh score that equals to or is greater than 5. In some embodiments, the subject has a Child-Pugh score that equals to or is greater than 7. In some embodiments, the subject has a Child-Pugh score that equals to or is greater than 10. In some embodiments, Child-Pugh score is not measured in a subject.

In some embodiments, the term “model for end-stage liver disease (MELD) score” is used for assessing liver disease. The term MELD is a prognostic scoring system based on laboratory parameters, used to predict 3-month mortality due to liver disease. See, e.g., Malinchoc M, Kamath P S, Gordon F D, et al. A model to predict poor survival in patients undergoing transjugular intrahepatic portosystemic shunts. Hepatology. 2000; 31(4):864-871, and Biggins S W, Kim W R, Terrault N A, et al. Evidence-Based Incorporation of Serum Sodium Concentration Into MELD. Gastroenterology. 2006; 130(6):1652-1660. In some embodiments, the MELD score can include the addition of serum sodium into the MELD score, i.e., the MELD-Na. See, e.g., Ruf A E, et al., “Addition of serum sodium into the MELD score predicts waiting list mortality better than MELD alone,” Liver Transpl. 2005 March; 11(3):336-43. MELD scores range from 6 to 40; the higher the score, the higher the 3-month mortality related to liver disease.

In some embodiments, the subject has an MELD score that equals to or is greater than 10, equals to or is greater than 15, equals to or is greater than 20, equals to or is greater than 25, equals to or is greater than 30, equals to or is greater than 35, or equals to or is greater than 40. In some embodiments, the subject has an MELD score that equals to or is greater than 10. In some embodiments, the subject has an MELD score that equals to or is greater than 20. In some embodiments, the subject has an MELD score that equals to or is greater than 25. In some embodiments, MELD score is not measured in a subject. In some embodiments, the subject has clinical or radiographic features of clinically significant portal hypertension. Such features may include the BAVENO VII criteria and include but are not limited to the presence of esophageal varices, collaterals imaging, liver stiffness >25 kPa, or liver stiffness >20 kPa and platelet count <150×10⁹/L, and/or a history of decompensation events. See e.g., Franchis et al., Baveno VII-Renewing consensus in portal hypertension. Journal of Hepatology. 2022; 76:959-974. In some aspects, a previously compensated subject has a first decompensation event, wherein the first decompensation event is chosen from variceal hemorrhage, hepatic encephalopathy, and/or ascites. In some aspects, the subject is already decompensated and has further decompensated events chosen from a) development of a second portal hypertension-driven decompensating event (ascites, variceal haemorrhage or hepatic encephalopathy) and/or jaundice; b) development of recurrent variceal bleeding, recurrent ascites (requirement of ≥3 large-volume paracenteses within 1 year), recurrent encephalopathy, development of spontaneous bacterial peritonitis and/or hepatorenal syndrome and/or acute kidney injury; c) and in subjects presenting with bleeding alone, development of ascites, encephalopathy, or jaundice after recovery from bleeding but not if these events occur around the time of bleeding. See Franchis at 967. In some embodiments, the subject has cirrhosis with features of portal hypertension. In some embodiments, the subject has cirrhosis with features of clinically significant portal hypertension. In some embodiments, the subject has compensated cirrhosis. In some events, the subject has decompensated cirrhosis.

In some embodiments, an effective amount or a therapeutically effect amount of zibotentan and dapagliflozin can be administered to the subject. The term “effective amount” or “therapeutically effective amount” means an amount of a designated compound sufficient to effectuate a desired physiological outcome in an individual in need of the compound. The effective amount may vary among individuals depending on the health and physical condition of the individual to be treated, the taxonomic group of the individuals to be treated, the formulation of the composition, assessment of the individual's medical condition, and other relevant factors.

In some embodiments, the composition can comprise various amounts of zibotentan. In some embodiments, the composition comprises a therapeutically effect amount of zibotentan. In some embodiments, the composition comprises about 0.1 mg to about 240 mg zibotentan. In some embodiments, the composition comprises about 0.2 mg to about 50 mg zibotentan. In some embodiments, the composition comprises about 0.5 mg to about 20 mg zibotentan. In some embodiments, the composition comprises about 1 mg to about 10 mg zibotentan. In some embodiments, the composition comprises about 0.1 mg, 0.25 mg, 0.5 mg, 0.75 mg, 1 mg, 2.5 mg, 5 mg, 6 mg, 7 mg, 8 mg, 9 mg, 10 mg, 12 mg, 15 mg, 20 mg, 30 mg, 60 mg, 120 mg, or 240 mg zibotentan. In some embodiments, the composition comprises about 1 mg, 2, mg, 2.5 mg, 3 mg, 4 mg, 5 mg, 6 mg, 7 mg, 7.5 mg, 8 mg, 9 mg, or 10 mg zibotentan. In some embodiments, the composition comprises about 1 mg zibotentan. In some embodiments, the composition comprises about 2.5 mg zibotentan. In some embodiments, the composition comprises about 5 mg zibotentan.

In some embodiments, the composition can comprise various amounts of dapagliflozin. In some embodiments, the composition comprises a therapeutically effect amount of dapagliflozin. In some embodiments, the composition comprises about 0.1 mg to about 100 mg dapagliflozin. In some embodiments, the composition comprises about 0.25 mg to about 50 mg dapagliflozin. In some embodiments, the composition comprises about 1 mg to about 10 mg dapagliflozin.

In some embodiments, the composition comprises about 2.5 mg, 3 mg, 4 mg, 5 mg, 6 mg, 7 mg, 8 mg, 9 mg, or 10 mg dapagliflozin. In some embodiments, the composition comprises about 2.5 mg dapagliflozin. In some embodiments, the composition comprises about 5 mg dapagliflozin. In some embodiments, the composition comprises about 10 mg dapagliflozin.

In some embodiments, the composition comprises a therapeutically effect amount of zibotentan and dapagliflozin. In some embodiments, the composition comprises about 0.2 mg to about 25 mg zibotentan and about 0.5 mg to about 25 mg dapagliflozin. In some embodiments, the composition comprises about 0.2 mg to about 25 mg zibotentan and about 1.25 mg to about 20 mg dapagliflozin. In some embodiments, the composition comprises about 0.2 mg to about 25 mg zibotentan and about 2.5 mg to about 10 mg dapagliflozin.

In some embodiments, the composition comprises about 0.5 mg to about 10 mg zibotentan and about 0.5 mg to about 25 mg dapagliflozin. In some embodiments, the composition comprises about 0.5 mg to about 10 mg zibotentan and about 1.25 mg to about 20 mg dapagliflozin. In some embodiments, the composition comprises about 0.5 mg to about 10 mg zibotentan and about 2.5 mg to about 10 mg dapagliflozin.

In some embodiments, the composition comprises about 1 mg to about 5 mg zibotentan and about 0.5 mg to about 25 mg dapagliflozin. In some embodiments, the composition comprises about 1 mg to about 5 mg zibotentan and about 1.25 mg to about 20 mg dapagliflozin. In some embodiments, the composition comprises about 1 mg to about 5 mg zibotentan and about 2.5 mg to about 10 mg dapagliflozin.

In some embodiments, the composition comprises about 0.2 mg, about 0.5 mg, about 1 mg, about 2 mg, about 2.5 mg, about 3 mg, about 4 mg, about 5 mg, about 6 mg, about 7 mg, about 8 mg, about 9 mg, about 10 mg zibotentan, and about 0.5 mg, about 1 mg, about 1.25 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg dapagliflozin.

In some embodiments, the composition comprises about 1 mg zibotentan and about 2.5 mg dapagliflozin. In some embodiments, the composition comprises about 1 mg zibotentan and about 5 mg dapagliflozin. In some embodiments, the composition comprises about 1 mg zibotentan and about 10 mg dapagliflozin.

In some embodiments, the composition comprises about 2.5 mg zibotentan and about 2.5 mg dapagliflozin. In some embodiments, the composition comprises about 2.5 mg zibotentan and about 5 mg dapagliflozin. In some embodiments, the composition comprises about 2.5 mg zibotentan and about 10 mg dapagliflozin.

In some embodiments, the composition comprises about 5 mg zibotentan and about 2.5 mg dapagliflozin. In some embodiments, the composition comprises about 5 mg zibotentan and about 5 mg dapagliflozin. In some embodiments, the composition comprises about 5 mg zibotentan and about 10 mg dapagliflozin.

In some embodiments, the ratio of zibotentan and the dapagliflozin can be fixed in the composition, e.g., when producing the composition to provide multiple fixed dosage form. For example, in some embodiments, the composition is in an amount to form greater than 1, greater than 10, or greater than 1000 dosage forms (e.g., capsules or tablets). In some embodiments, the skilled artisan will appreciate that any of the amounts listed above for a “composition” can apply to a single “dosage form.” Thus, e.g., the disclosure of “the composition comprises about 5 mg zibotentan and about 2.5 mg dapagliflozin” includes a single dosage form comprising about 5 mg zibotentan and about 2.5 mg dapagliflozin.

In some embodiments, a weight ratio between the zibotentan and the dapagliflozin in the composition is about 1:50 to about 10:1, about 1:20 to about 4:1, or about 1:10 to about 2:1. In some embodiments, a weight ratio between zibotentan and dapagliflozin in the composition is about 1:2.5, about 1:5, or about 1:10. In some embodiments, a weight ratio between zibotentan and dapagliflozin in the composition is about 1:1 to about 1:2, or about 1:4. In some embodiments, a weight ratio between zibotentan and dapagliflozin in the composition is about 2:1, about 1:1, or about 1:2.

The term “administration” or “administering” refers to routes of introducing a compound or composition provided herein to an individual to perform its intended function. An example of a route of administration that can be used includes but is not limited to oral administration or parenteral administration, such as subcutaneous, intravenous, or intramuscular injection or infusion. In some embodiments, the route of administration is oral administration. In some embodiments, the methods described herein are performed via oral administration, e.g., an oral dosage form. In some embodiments, the oral dosage from is a capsule or tablet.

As used herein, dosage form or unit dose is a pharmaceutical drug product in the form in which it is marketed for use, with a specific mixture of active ingredients and inactive components (excipients), in a particular configuration (such as a capsule shell, for example), and apportioned into a particular dose. Various dosage forms may exist for a single particular drug, since different medical conditions can warrant different routes of administration. Dosage forms may include, for example, oral dosage form, ophthalmic dosage form, inhalation, injection, parenteral, topical, or suppository. Oral dosage forms may include, for example, pill in the form of tablet or capsule, syrups, specialty tablet like buccal, sublingual, or orally-disintegrating, thin film, liquid solution or suspension, powder or liquid or solid crystals, natural or herbal plant, seed, or food of sorts, pastes, or buccal film.

In some embodiments, the zibotentan is in an oral dosage form. In some embodiments, the zibotentan oral dosage form is a capsule or a tablet. In some embodiments, the dapagliflozin is in an oral dosage form. In some embodiments, the dapagliflozin oral dosage form is a capsule or a tablet. In some embodiments, the composition, i.e., the composition comprising zibotentan and dapagliflozin, is in an oral dosage form. In some embodiments, the zibotentan/dapagliflozin oral dosage form is a capsule or tablet. In some embodiments, the zibotentan/dapagliflozin oral dosage form is a capsule.

In some embodiments, the composition is administered to the subject once to three times daily. In some embodiments, the composition is administered to the subject twice daily. In some embodiments, the composition is administered to the subject once daily.

As used herein, the term daily refers to a 24-hour period. For example, in some embodiments, the compositions of the present disclosure can be administered in the morning, in the evening or mid-day. In some embodiments, the compositions of the present disclosure can be administered after consuming food (e.g., after a meal), or before consuming food (e.g., before a meal, i.e., fasting conditions). The dosing frequency of the composition can be determined by the one of ordinary skill in the art. In some embodiments, the compositions of the present disclosure can be administered in 8-hour intervals, 12 hour intervals, or 24 hour intervals. In some embodiments, the composition is administered daily, twice daily or three times daily. In some embodiments, the composition is administered weekly, twice weekly or three times weekly. In some embodiments, the composition is administered monthly, twice monthly or three times monthly. In some embodiments, the composition is administered not more than once a week, once every two weeks, once every three weeks, once every 4 weeks, once every five weeks once every six weeks, or once every 7 weeks. In some embodiments, the composition is administered “as needed”, e.g., when symptoms associated with a liver cirrhosis decompensation even occurs, until the symptoms are alleviated.

In some embodiments, the composition is administered to the subject once to three times daily for at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, or more. In certain embodiments, the composition is administered to the subject once daily for 3 weeks, or 6 weeks, or 12 weeks, or 16 weeks, or 18 weeks, or more. In some embodiments, the composition is administered to the subject at least one to three times daily for an extended period of time greater than 16 weeks (32 weeks, 64 weeks, 1 year, 5 years, or longer) depending on the requirements of the subject(s).

In some embodiments, the disclosure provides a method for decreasing portal venous pressure in a subject. In some embodiments, the reduction in portal venous pressure of the subject is determined by any of the methods described herein (e.g., reduction of HVPG), using any of the methods and compositions described herein.

In some embodiments, the subject has a hepatic venous pressure gradient (HVPG) response; and the HVPG is decreased at least 10% after administration of the composition to the subject once to three times daily for 6 weeks relative to a baseline HVPG of the subject. In some embodiments, the baseline HVPG refers to the HVPG of the subject immediately before administration of the composition. Thus, the for a subject having an elevated HVPG of 10 mmHg, the phrase “the HVPG is decreased at least 10% after administration of the composition to the subject,” would indicate a reduction of at least 1 mmHg (i.e., 10% of 10 mmHg) in the subject after administration of compositions described herein. In some embodiments, the HVPG of the subject is decreased at least 15%, at least 20%, at least 25%, at least 30%, at least 35%, or at least 40% after administration of the composition to the subject. In some embodiments, the HVPG of the subject is decreased about 10% to about 50%, about 20% to about 40%, or about 20% to about 30% after administration of the composition to the subject.

In some embodiments, the HPVG in the subject is decreased 1 mmHg to 8 mmHg, 1 mmHg to 7 mmHg, 1 mmHg to 6 mmHg, 2 mmHg to 5 mmHg or 3 mmHg to 4 mmHg after administration of the composition to the subject. In some embodiments, the HVPG of the subject is decreased to about less than 5 mmHg, less than 6 mmHg, less than 7 mmHg or less than 8 mmHg after administration of the composition to the subject. In some embodiments, the HVPG measurement is performed after administration of the composition to the subject once to three times daily for 1 week, 2 weeks, 3 weeks, 4 weeks, 5 weeks, 6 weeks, or more. In some embodiments, the HVPG measurement is performed after administration of the composition to the subject once to three times daily for 6 weeks or more. In some embodiments, the HVPG measurement is performed after administration of the composition to the subject once daily for 6 weeks or more.

In some embodiments, the endpoint for HVPG can also be determined according the HVPG measurement in mmHg. In some embodiments, the subject has a reduction in HVPG; and the HVPG is less than or equal to 12 mmHg after administration of the composition to the subject relative to a baseline HVPG of the subject. In some embodiments, the HVPG response of the subject is less than or equal to 11 mmHg, 10 mmHg, 9 mmHg, 8 mmHg, 7 mmHg, 6 mmHg, or 5 mmHg after administration of the composition to the subject. In some embodiments, the HVPG measurement is performed after administration of the composition to the subject once to three times daily for 6 weeks for more.

In some embodiments, the effect of administrating the composition can also be evaluated using endpoints of secondary objectives. In some embodiments, the subject has one or more of the following outcomes after administration of the composition to the subject: (i) at least 5% decrease in body weight; (ii) at least 5% reduction in body water volume; (iii) at least 5% reduction in body fat mass; (iv) systolic blood pressure is reduced to less than 130; (v) diastolic blood pressure is reduced to less than 90; and (vi) at least 5% reduction in accumulated additional loop-diuretic equivalents use.

In some embodiments, the subject has one or more of the following outcomes after administration of the composition to the subject: (i) at least 10% decrease in body weight; (ii) at least 10% reduction in body water volume; (iii) at least 10% reduction in body fat mass; (iv) systolic blood pressure is reduced to less than 120; (v) diastolic blood pressure is reduced to less than 80; and (vi) at least 10% reduction in accumulated additional loop-diuretic equivalents use.

In some embodiments, the subject has one or more of the following outcomes after administration of the composition to the subject: (i) at least 15% decrease in body weight; (ii) at least 15% reduction in body water volume; (iii) at least 15% reduction in body fat mass; (iv) systolic blood pressure is reduced to less than 110; (v) diastolic blood pressure is reduced to less than 70; and (vi) at least 15% reduction in accumulated additional loop-diuretic equivalents use.

In some embodiments, the methods of treating liver cirrhosis can be continued until one or more of the end points of secondary objectives is met.

In some embodiments, the subject has at least 1%, at least 2%, at least 3%, at least 4%, at least 5%, at least 6%, at least 7%, at least 8%, at least 9%, at least 10%, at least 15%, at least 20%, at least 25%, or at least 30% reduction in body weight after administration of the composition to the subject once to three times daily for 6 weeks.

In some embodiments, the subject has at least 1%, at least 2%, at least 3%, at least 4%, at least 5%, at least 6%, at least 7%, at least 8%, at least 9%, at least 10%, at least 15%, at least 20%, at least 25%, or at least 30% reduction in body water volume after administration of the composition to the subject once to three times daily for 6 weeks.

In some embodiments, the subject has at least 1%, at least 2%, at least 3%, at least 4%, at least 5%, at least 7%, at least 10%, at least 15%, at least 20%, at least 25%, or at least 30% reduction in body fat mass after administration of the composition to the subject once to three times daily for 6 weeks.

In some embodiments, systolic blood pressure of the subject is reduced to less than 140, less than 135, less than 130, less than 125, or less than 120, after administration of the composition to the subject once to three times daily for 6 weeks.

In some embodiments, diastolic blood pressure of the subject t is reduced to less than 90, less than 88, less than 86, less than 84, less than 82, or less than 80, after administration of the composition to the subject once to three times daily for 6 weeks.

In some embodiments, the subject has at least 1%, at least 2%, at least 3%, at least 4%, at least 5%, at least 6%, at least 7%, at least 8%, at least 9%, at least 10%, at least 15%, at least 20%, at least 25%, or at least 30% reduction in accumulated additional loop-diuretic equivalents use after administration of the composition to the subject once to three times daily for 6 weeks.

The methods described herein are for the treatment of liver cirrhosis. Liver cirrhosis can result from disease (e.g., viral infections) or conditions (e.g., chronic alcoholism, obesity, etc.), whereby scar tissue forms on the liver making it difficult for the liver to function. In some embodiments, the liver cirrhosis results from viral hepatitis, alcoholic liver disease, hepatic steatosis, non-alcoholic fatty liver disease (NAFLD), non-alcoholic steatohepatitis (NASH), or combinations thereof. In some embodiments, the liver cirrhosis results from autoimmune hepatitis, primary sclerosing cholangitis, primary biliary cholangitis, haemochromatosis, Wilson's disease, biliary atresia, alpha-1-antitrypsin deficiency and cystic fibrosis related liver cirrhosis or combinations thereof. A “subject in need” of treatment can include any subject with liver cirrhosis, independent of the cause of the liver cirrhosis. Additionally, in some embodiment a “subject in need” of treatment can include any subject with liver cirrhosis, even if the subject is not exhibiting decompensation events.

In certain embodiments, in addition to the methods of treating cirrhosis as described herein, the subject can also be treated with an additional standard of care (SoC) therapy. Here the term “standard of care” means a diagnostic and treatment process that a clinician should follow for a certain type of patient, illness, or clinical circumstance. In some embodiments, the standard of care of cirrhosis may include, for example, a low-sodium diet and medication (e.g., beta blockers such as carvedilol) to reduce portal blood pressure, and diuretics such as spironolactone or furosemide to prevent fluid buildup in the body, draining of fluid or surgery to relieve pressure when the subject experiences severe fluid buildup, treating with antibiotics, or other treatments associated with cirrhosis. In some embodiments, the method of the present disclosure further comprises: (i) administration of an additional act agent; (ii) administration of a low sodium diet; or (iii) combinations thereof.

In certain aspects, the present disclosure provides an oral dosage form comprising zibotentan and dapagliflozin. The present disclosure provides that combinations of therapeutically effective amounts of zibotentan and dapagliflozin when taken orally together, e.g., in the same dosage form, can be useful in the treatment of liver cirrhosis. In some embodiments, the oral dosage form comprises: (i) about 0.25 mg to about 20 mg zibotentan; (ii) about 0.5 mg to about 40 mg dapagliflozin; and (iii) a pharmaceutical acceptable excipient.

In some embodiments, the oral dosage form comprises about 0.5 mg to about 10 mg zibotentan, and about 1 mg to about 20 mg dapagliflozin. In some embodiments, the oral dosage form comprises about 1.0 mg to about 5 mg zibotentan, and about 2.5 mg to about 10 mg dapagliflozin. In some embodiments, the oral dosage form comprises about 2.5 mg to about 5 mg zibotentan, and about 2.5 mg to about 10 mg dapagliflozin.

In some embodiments, the oral dosage form comprises about 1 mg zibotentan and about 2.5 mg dapagliflozin. In some embodiments, the oral dosage form comprises about 1 mg zibotentan and about 5 mg dapagliflozin. In some embodiments, the single oral dosage form comprises about 1 mg zibotentan and about 10 mg dapagliflozin. In some embodiments, the oral dosage form comprises about 2.5 mg zibotentan and about 2.5 mg dapagliflozin. In some embodiments, the oral dosage form comprises about 2.5 mg zibotentan and about 5 mg dapagliflozin. In some embodiments, the oral dosage form comprises about 2.5 mg zibotentan and about 10 mg dapagliflozin. In some embodiments, the oral dosage form comprises about 5 mg zibotentan and about 5 mg dapagliflozin. In some embodiments, the oral dosage form comprises about 5 mg zibotentan and about 10 mg dapagliflozin.

In some embodiments, the disclosure provides a kit comprising the compositions and/or dosage forms described herein, for use in the methods described herein. In some embodiments, the kit comprises: (a) an oral dosage form comprising about 0.25 mg to about 20 mg zibotentan, and a pharmaceutically acceptable excipient; and (b) an oral dosage form comprising about 0.5 mg to about 40 mg dapagliflozin, and a pharmaceutically acceptable excipient. In some embodiments, the kit comprises: (a) multiple dosage forms comprising zibotentan, wherein each individual oral dosage form of zibotentan comprises about 0.25 mg to about 20 mg zibotentan, and a pharmaceutically acceptable excipient; and (b) multiple dosage forms comprising dapagliflozin, wherein each oral dosage form of dapagliflozin comprises about 0.5 mg to about 40 mg dapagliflozin, and a pharmaceutically acceptable excipient.

In some embodiments, the kit comprises: (a) an oral dosage form comprising about 2.5 mg zibotentan, and a pharmaceutically acceptable excipient; and (b) an oral dosage form comprising about 5 mg dapagliflozin, and a pharmaceutically acceptable excipient. In some embodiments, the kit comprises: (a) multiple dosage forms comprising zibotentan, wherein each individual oral dosage form of zibotentan comprises about 2.5 mg zibotentan, and a pharmaceutically acceptable excipient; and (b) multiple dosage forms comprising dapagliflozin, wherein each oral dosage form of dapagliflozin comprises about 5 mg dapagliflozin, and a pharmaceutically acceptable excipient.

In some embodiments, the kit comprises 1 to 31 dosage forms comprising zibotentan dosage forms, and 1 to 31 dosage forms comprising dapagliflozin. In some embodiments, the kit comprises (a) a number of dosage forms comprising zibotentan sufficient for treatment of the subject for 1 week, 1 month, 2 months, or 3 months, and (b) a number of dosage forms comprising dapagliflozin sufficient for treatment of the subject for 1 week, 1 month, 2 months, or 3 months. In some embodiments, the kit comprises instructions for administering the dosage forms found in the kit. In some embodiments, the kit comprises packaging for the transportation and storage of the oral dosage forms. In some embodiments, the packaging separates the dosage forms of zibotentan from the dosage forms of dapagliflozin.

In certain aspects, the present disclosure relates to a method of reducing portal venous pressure in a subject in need thereof. In some embodiments, the method comprises administering to the subject a composition comprising a therapeutically effective amount of zibotentan and dapagliflozin. In some embodiments, the method comprises administering a composition (e.g., capsule or tablet) comprising about 0.25 mg to about 20 mg dapagliflozin and about 0.5 mg to about 10 mg zibotentan. In some embodiments, the composition comprises about 1 mg, 2.5 mg, or 5 mg zibotentan, and about 2.5 mg, 5 mg, or 10 mg dapagliflozin. In some embodiments, HVPG is reduced at least 20% after administration of the composition to the subject. In some embodiments, the HVPG of the subject is decreased at least 25% after administration of the composition to the subject. In some embodiments, the HVPG of the subject is decreased at least 30% after administration of the composition to the subject. In some embodiments, the HVPG of the subject is decreased at least 35% after administration of the composition to the subject. In some embodiments, the HVPG of the subject is decreased at least 40% after administration of the composition to the subject. In some embodiments, the subject has a reduction of hepatic venous pressure gradient (HVPG) to less than or equal to 12 mmHg after administration of the composition to the subject relative to a baseline HVPG of the subject. In some embodiments, the HVPG of the subject is less than or equal to 10 mmHg after administration of the composition to the subject. In some embodiments, the HVPG of the subject is less than or equal to 8 mmHg after administration of the composition to the subject. In some embodiments, the HVPG of the subject is reduced down to less than or equal to 5 mmHg after administration of the composition to the subject. In some embodiments, the subject's HVPG is reduced at least 1.0 mmHg after administration of the composition to the subject. In some embodiments, the subject's HVPG is reduced at least 1.5 mmHg after administration of the composition to the subject. In some embodiments, the subject's HVPG is reduced at least 2.0 mmHg after administration of the composition to the subject. In some embodiments, the subject's HVPG is reduced at least 2.5 mmHg after administration of the composition to the subject.

In certain aspects, the present disclosure relates to a method of reducing the Child Pugh score in a subject in need thereof to a score lower than the Child Pugh score of the subject prior to administration. In some embodiments, the method comprises administering to the subject a composition comprising a therapeutically effective amount of zibotentan and dapagliflozin. In some embodiments, the method comprises administering a composition (e.g., capsule or tablet) comprising about 0.25 mg to about 20 mg dapagliflozin and about 0.5 mg to about 10 mg zibotentan. In some embodiments, the composition comprises about 1 mg, 2.5 mg, or 5 mg zibotentan, and about 2.5 mg, 5 mg, or 10 mg dapagliflozin. In some embodiments, the Child Pugh score is reduced by at least 1 point, 2 points, 3 points, 4 points, 5 points, 6 points, 7 points, 8 points, 9 points, 10 points or more. In at least one embodiment, the Child Pugh score is reduced by at least 5 points or more. In at least one embodiment, the subject's Child Pugh score is reduced in severity to a different class (e.g., Class B reduced to Class A after administration of the composition to the subject or Class C reduced to Class B after administration of the composition to the subject).

In certain aspects, the present disclosure relates to a method of reducing the MELD score of a subject in need thereof to a score lower than the MELD score of the subject prior to administration. In some embodiments, the method comprises administering to the subject a composition comprising a therapeutically effective amount of zibotentan and dapagliflozin. In some embodiments, the method comprises administering a composition (e.g., capsule or tablet) comprising about 0.25 mg to about 20 mg dapagliflozin and about 0.5 mg to about 10 mg zibotentan. In some embodiments, the composition comprises about 1 mg, 2.5 mg, or 5 mg zibotentan, and about 2.5 mg, 5 mg, or 10 mg dapagliflozin. In some embodiments, the MELD score is reduced by at least 1 point, 2 points, 3 points, 4 points, 5 points, 6 points, 7 points, 8 points, 9 points, 10 points, 11 points, 12 points, 13 points, 14 points, 15 points, 16 points, 17 points, 18 points, 19 points, 20 points or more. In at least one embodiment, the MELD score is reduced by at least 5 points or at least 10 points or at least 15 points or at least 20 points or more.

In certain aspects, the present disclosure relates to a method of treating liver cirrhosis in a subject in need thereof, the method comprises administering to the subject a composition comprising a therapeutically effective amount of zibotentan and dapagliflozin. In some embodiments, the method comprises administering a composition (e.g., capsule or tablet) comprising about 0.25 mg to about 20 mg dapagliflozin and about 0.5 mg to about 10 mg zibotentan. In some embodiments, the composition comprises about 1 mg, 2.5 mg, or 5 mg zibotentan, and about 2.5 mg, 5 mg, or 10 mg dapagliflozin. In some embodiments, the cirrhosis may be compensated or decompensated. In some aspects, the subject may be compensated or decompensated. Decompensated subjects may either be in stable condition or unstable where they have recently suffered recurrent decompensation, e.g., during the last 1-3-months' time period.

In certain aspects, the present disclosure relates to a method of treating portal hypertension in a subject with liver cirrhosis in need thereof, the method comprises administering to the subject a composition comprising a therapeutically effective amount of zibotentan and dapagliflozin. In some embodiments, the method comprises administering a composition (e.g., capsule or tablet) comprising about 0.25 mg to about 20 mg dapagliflozin and about 0.5 mg to about 10 mg zibotentan. In some embodiments, the composition comprises about 1 mg, 2.5 mg, or 5 mg zibotentan, and about 2.5 mg, 5 mg, or 10 mg dapagliflozin. In some embodiments, the method reduces the subject's HVPG by at least 20% after administration of the composition to the subject. In some embodiments, the HVPG of the subject is decreased at least 25% after administration of the composition to the subject. In some embodiments, the HVPG of the subject is decreased at least 30% after administration of the composition to the subject. In some embodiments, the HVPG of the subject is decreased at least 35% after administration of the composition to the subject. In some embodiments, the HVPG of the subject is decreased at least 40% after administration of the composition to the subject. In some embodiments, the subject has a reduction of hepatic venous pressure gradient (HVPG) to less than or equal to 12 mmHg after administration of the composition to the subject relative to a baseline HVPG of the subject. In some embodiments, the HVPG of the subject is less than or equal to 10 mmHg after administration of the composition to the subject. In some embodiments, the HVPG of the subject is less than or equal to 8 mmHg after administration of the composition to the subject. In some embodiments, the HVPG of the subject is reduced down to less than or equal to 5 mmHg after administration of the composition to the subject. In some embodiments, the subject's HVPG is reduced at least 1.0 mmHg after administration of the composition to the subject. In some embodiments, the subject's HVPG is reduced at least 1.5 mmHg after administration of the composition to the subject. In some embodiments, the subject's HVPG is reduced at least 2.0 mmHg after administration of the composition to the subject. In some embodiments, the subject's HVPG is reduced at least 2.5 mmHg after administration of the composition to the subject.

In certain aspects, the present disclosure relates to a method of treating clinically significant portal hypertension in a subject with liver cirrhosis in need thereof, the method comprises administering to the subject a composition comprising a therapeutically effective amount of zibotentan and dapagliflozin. In some embodiments, the method comprises administering a composition (e.g., capsule or tablet) comprising about 0.25 mg to about 20 mg dapagliflozin and about 0.5 mg to about 10 mg zibotentan. In some embodiments, the composition comprises about 1 mg, 2.5 mg, or 5 mg zibotentan, and about 2.5 mg, 5 mg, or 10 mg dapagliflozin. In some embodiments, the method reduces the subject's HVPG by at least 20% after administration of the composition to the subject. In some embodiments, the HVPG of the subject is decreased at least 25% after administration of the composition to the subject. In some embodiments, the HVPG of the subject is decreased at least 30% after administration of the composition to the subject. In some embodiments, the HVPG of the subject is decreased at least 35% after administration of the composition to the subject. In some embodiments, the HVPG of the subject is decreased at least 40% after administration of the composition to the subject. In some embodiments, the subject has a reduction of hepatic venous pressure gradient (HVPG) to less than or equal to 12 mmHg after administration of the composition to the subject relative to a baseline HVPG of the subject. In some embodiments, the HVPG of the subject is less than or equal to 10 mmHg after administration of the composition to the subject. In some embodiments, the HVPG of the subject is less than or equal to 8 mmHg after administration of the composition to the subject. In some embodiments, the HVPG of the subject is reduced down to less than or equal to 5 mmHg after administration of the composition to the subject. In some embodiments, the subject's HVPG is reduced at least 1.0 mmHg after administration of the composition to the subject. In some embodiments, the subject's HVPG is reduced at least 1.5 mmHg after administration of the composition to the subject. In some embodiments, the subject's HVPG is reduced at least 2.0 mmHg after administration of the composition to the subject. In some embodiments, the subject's HVPG is reduced at least 2.5 mmHg after administration of the composition to the subject.

In certain aspects, the present disclosure relates to a method of reducing the risk of variceal haemorrhage and/or death or preventing and/or delaying progression of variceal haemorrhage and/or death, in a subject in need thereof, the method comprising administering to the subject a composition comprising a therapeutically effective amount of zibotentan and dapagliflozin. In some embodiments, the method comprises administering a composition (e.g., capsule or tablet) comprising about 0.25 mg to about 20 mg dapagliflozin and about 0.5 mg to about 10 mg zibotentan. In some embodiments, the composition comprises about 1 mg, 2.5 mg, or 5 mg zibotentan, and about 2.5 mg, 5 mg, or 10 mg dapagliflozin.

In certain aspects, the present disclosure provides a method of reducing the risk of cirrhosis decompensation or preventing and/or delaying progression of cirrhosis decompensation and death, in a subject in need thereof, the method comprising administering to the subject a composition comprising a therapeutically effective amount of zibotentan and dapagliflozin. In some embodiments, the method comprises administering a composition (e.g., capsule or tablet) comprising about 0.25 mg to about 20 mg dapagliflozin and about 0.5 mg to about 10 mg zibotentan. In some embodiments, the composition comprises about 1 mg, 2.5 mg, or 5 mg zibotentan, and about 2.5 mg, 5 mg, or 10 mg dapagliflozin. In some embodiments, the method reduces the risk of developing new variceal hemorrhage or worsening of hepatic encephalopathy or worsening of ascites.

In certain aspects, the present disclosure relates to a method of treating ascites and/or preventing or reducing the formation of ascites and/or delaying progression of ascites, preventing complications of ascites (e.g., bacterial peritonitis, hepato-pleural effusion, or refractory ascites) and/or reducing the volume of ascites in a subject in need thereof. In some embodiments, the method comprises administering to the subject a composition comprising a therapeutically effective amount of zibotentan and dapagliflozin. In some embodiments, the method comprises administering a composition (e.g., capsule or tablet) comprising about 0.25 mg to about 20 mg dapagliflozin and about 0.5 mg to about 10 mg zibotentan. In some embodiments, the composition comprises about 1 mg, 2.5 mg, or 5 mg zibotentan, and about 2.5 mg, 5 mg, or 10 mg dapagliflozin. In some embodiments, the method prevents the development of Grade 1 ascites. In some embodiments, the method delays or prevents the progression of ascites from Grade 1 to Grade 2 or Grade 2 to Grade 3. In some embodiments, the method reduces the severity of ascites, for example, from Grade 3 to Grade 2 or Grade 2 to Grade 1 or Grade 1 to absence of ascites. “Grade 1” ascites is understood to mean mild ascites and detectable only by imaging or ultrasound examination. “Grade 2” ascites is understood to mean moderate ascites and manifested by moderate symmetrical distension of the abdomen. “Grade 3” ascites is understood to mean large or gross ascites distinguished by marked abdominal distension.

In certain aspects, the present disclosure provides a method of reducing the composite of time to variceal haemorrhage, encephalopathy, worsening or complications of ascites (e.g., bacterial peritonitis, hepato-pleural effusion, or refractory ascites) and all-cause mortality in a subject in need thereof, the method comprising administering to the subject a composition comprising a therapeutically effective amount of zibotentan and dapagliflozin. In some embodiments, the method comprises administering a composition (e.g., capsule or tablet) comprising about 0.25 mg to about 20 mg dapagliflozin and about 0.5 mg to about 10 mg zibotentan. In some embodiments, the composition comprises about 1 mg, 2.5 mg, or 5 mg zibotentan, and about 2.5 mg, 5 mg, or 10 mg dapagliflozin.

The term “prevent” refers to stopping the onset, development or progression of a disease, disorder, or condition for a period of time from minutes to indefinitely. Delaying refers to forestalling the onset, development or progression of a disease, disorder, or condition for a period of time from minutes to indefinitely. The term “reducing” or “reduce” means to bring down to a smaller extent, size, amount, or number. Thus, a “reduction in the risk of cirrhosis decompensation” would include reducing the incidence and/or magnitude of cirrhosis decompensation.

In any of the methods above, the reduction or prevention or treatment may be absolute or in relation to a placebo or standard of care agent or monotherapy agent (i.e., dapagliflozin monotherapy or zibotentan monotherapy). Standard statistical considerations will apply (e.g., relative risk ratio, hazard ratio, odds ratio, etc.)

Also disclosed herein are methods of treating a subject having cirrhosis with signs of clinically significant portal hypertension with or without decompensation, comprising:
a) Determining concentrations of multiple clinical pathological markers of the subject;
b) Providing, to a machine learning model, the determined values of the multiple clinical pathological markers of the subject;
c) Receiving, from the machine learning model, a prediction of whether the patient has a likelihood of progression to a condition; and
d) Administering to the subject a composition comprising a therapeutically effective amount of zibotentan and dapagliflozin if the subject indicates a likelihood of progression to the condition.

In the methods disclosed above, the clinical pathological markers may be one or more of demographic values (age, gender, race or ethnicity), comorbidity values (diabetes status or obesity), vital sign values (body mass index, blood pressure, or heart rate), blood test values, or disease progression values. In addition, markers may include a history of decompensation or compensated cirrhosis with features of clinically significant portal hypertension including, but not limited to, oesophageal varices, splenomegaly, low platelet count, liver stiffness >25 kPa or >20 kPa+platelet count <150×10⁹. In the methods above, treatment may begin in subjects with compensated cirrhosis with features of portal hypertension and/or in decompensated cirrhosis subjects with or without a recent decompensation event (e.g., within the last 2-3 months).

All references cited herein, including patents, patent applications, papers, textbooks and the like, and the references cited therein, to the extent that they are not already, are hereby incorporated herein by reference in their entirety.

### EXAMPLE 1: Zibotentan and Dapagliflozin Fixed Dose Study

A Two-Part Phase (Part A and Part B) randomized, double-blind, placebo-controlled, parallel group dose ranging study will be conducted to assess the efficacy, safety, and tolerability of the combination of zibotentan and dapagliflozin, and dapagliflozin monotherapy versus placebo in participants with cirrhosis with features of portal hypertension. Particularly, Part A assesses the efficacy, safety, and tolerability of the combination of 2.5 mg zibotentan and 10 mg dapagliflozin versus placebo in participants with Child-Pugh A cirrhosis with features of portal hypertension and with no history of decompensation events. The safety profile is determined to be acceptable at the conclusion of Part A. Part B investigates efficacy, safety, and tolerability of 1 mg, 2.5 mg, or 5 mg zibotentan combined with 10 mg dapagliflozin and of 10 mg dapagliflozin monotherapy versus placebo in participants with cirrhosis with features of portal hypertension. Part B includes a broader range of Child-Pugh A and Child-Pugh B cirrhosis participants, including those with more severe disease, a history of decompensation events, or current ascites.

### 1. Overall Design of the Example

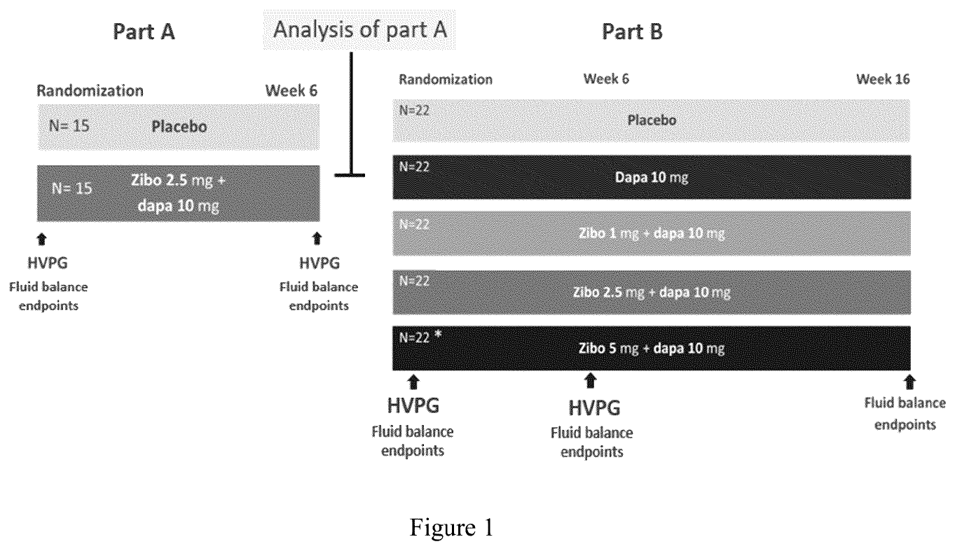

FIG. 1 shows study design of the study as outlined in Example 1.

### 1.1 Period

Part A 1 is a parallel group, 2 arm study that is blinded to the participants, investigators, and sponsor. Part B is a parallel group, 5 arm study that is blinded to the participants, investigators, and sponsor. Part A and Part B each includes a screening period, a treating period, and a follow-up period.

The screening period is up to 6 weeks; to confirm participant eligibility and collect baseline data on vital signs; clinical chemistry, haematology, and urinalysis; and ECHO/cardiac MRI (Part B only). Data is collected at two time periods (Visits 1 and 2). At Visit 2, eligibility criteria are assessed, safety laboratory data are re-verified, and participants are provided digital medical devices to measure their body weight starting before the randomisation visit and every day at home. At Visit 2, baseline HVPG recording are done and quality of the HVPG recording are judged by central reader of HVPG. Only the participants having good enough quality of the HVPG to accurately determine HVPG are then subjected to randomisation.

The treatment period is 6 weeks for Part A and 16 weeks for Part B. During the treatment period, data is collected from participants 5 times (Part A) or 8 times (Part B). At Visit 3 (Day 1), participants are randomised and take the first dose of the study intervention. At each data collection during the treatment period (except at days of HVPG assessment and VCTE/Fibroscan assessment, if visit procedures are separated on different days), participants take the once daily dose at the study centre and provide a pre dose blood sample for PK analysis and other laboratory samples before intake of study intervention. At Part A Visit 6 (or Visit 7) and Part B Visit 8 (or Visit 9), participants provide pre and post dose blood samples for PK analysis over about 5 hours.

The follow-up period is 2 weeks. Participants don't take the study intervention during this period and would return for follow-up assessments approximately 2 weeks after their last dose of study intervention.

The study intervention is a once daily dose of the assigned study intervention (oral capsules and tablets) during the treatment period (6 weeks for Part A, 16 weeks for Part B), in addition to their standard of care therapy. Total study duration for participants (including the screening and follow up period) is approximately 13 weeks for Part A and 24 weeks for Part B.

Table 1 shows Part A schedule of activities.

TABLE 1
Part A Schedule of Activities

| | | Treatment period | | | | | Follow-up/early |
|---|---|---|---|---|---|---|---|
| Screening period | | Randomisation | Visits | | | | termination |
| Visit number | | | | | | | |
| 1 | 2 | 3 | 4 | 5 | 6 | 7 | 8 |
| Week | | | | | | | |
| −6 | −1^{a} | 0^{a} | 1 | 2 | 3 | 6^{a} | 8 |
| Study day | | | | | | | |
| −35 | −5 | 1 | 8 | 15 | 22 | 43 | 57 |

Visit window (days)

| | | | | | |
|---|---|---|---|---|---|
| ±4 | ±2 | ±2 | ±2 | ±5 | ±3 |

| | | | | |
|---|---|---|---|---|
| Informed consent | X | | | |
| Inclusion/exclusion | X | X | X | |
| criteria | | | | |
| Alcohol breath test | X | | X | X |
| Screen for drugs of abuse | X | | | |
| AUDIT questionnaire | X | | | |
| Enrolment in IRT/RTSM | X | | | |
| Randomization in | | | X | |
| IRT/RTSM ^{m} | | | | |

Routine clinical procedures

| | | | | | | | | |
|---|---|---|---|---|---|---|---|---|
| Demography and | X | | | | | | | |
| baseline characteristics | | | | | | | | |
| (including smoking | | | | | | | | |
| history and alcohol | | | | | | | | |
| consumption) | | | | | | | | |
| Medical/surgical history | X | | | | | | | |
| Physical examination | X | | X | X ^{c} | X ^{c} | X ^{c} | X ^{c} | X |
| Concomitant Medication | X | | X | X | X | X | X | X |
| Body weight | X | | X | X | X | X | X | X |
| Height, BMI calculation | X | | | | | | | |
| ECG | X | X ^{q} | X (predose, | X | X | X | X ^{q} | X |
| | | | 2 hours | | | | | |
| | | | postdose) | | | | | |
| FSH (females only) | X ^{d} | | | | | | | |
| Pregnancy tests (females | X | | X | | | | | X |
| only; serum or urine) | (serum) | | | | | | | |

Study intervention

| | | | | | |
|---|---|---|---|---|---|
| Study intervention | X | | | X | |
| dispensed | | | | | |
| Study intervention | | X | X | X | X |
| accountability (counting | | | | | |
| remaining tablets and | | | | | |
| capsules) | | | | | |
| Study intervention intake ⁿ | X | X | X | X | X |

Efficacy assessments

| HVPG ^{e} | X ^{b, j} | | | | | X ^{b,j} | |
|---|---|---|---|---|---|---|---|
| VCTE/Fibroscan ^{p} | | X ^{b} | | | | X ^{b} | |
| Daily digital body weight | X | Daily home measurement using a digital device | | | | | |
| measurement | | from baseline to end of follow-up. | | | | | |
| Bioimpedance | X | X | X | X | X | X | X |
| spectroscopy | | | | | | | |

Safety assessments

| | | | | | | | | |
|---|---|---|---|---|---|---|---|---|
| Adverse event review ^{k} | X | X | X | X | X | X | X | X |
| | SAEs | SAEs | | | | | | |
| | only | only | | | | | | |
| Pulse and orthostatic test | X | X | X (predose, 60, | X | X | X | X | X |
| (supine and standing | | | 120, 240 | | | | | |
| blood pressure) | | | minutes) | | | | | |
| Body temperature | X | X | X | X | X | X | X | X |
| Safety clinical chemistry, | X | X | X ^{f} | X ^{f} | X ^{f} | X ^{f} | X ^{f} | X |
| electrolyte, coagulation | | | | | | | | |
| and haematology | | | | | | | | |
| HIV Serology | X | | | | | | | |
| Other safety test: | X | X | X ^{f} | X ^{f} | X ^{f} | X ^{f} | X ^{f} | X |
| Glutamate | | | | | | | | |
| Dehydrogenase (GLDH), | | | | | | | | |
| Serum Osmolality, BNP | | | | | | | | |
| and NT-pro BNP | | | | | | | | |

PK assessments

| | | | | | |
|---|---|---|---|---|---|
| Pre-dose PK plasma | X | X | X | X | X ^{g} |
| sample ^{o} | | | | | |
| PK profile | | | X ^{h,i} | | |

Exploratory assessments

| | | | | | | | | |
|---|---|---|---|---|---|---|---|---|
| MELD score calculation | X | X | X | X | X | X | X | X |
| eGFR calculation | x | X | X | X | x | x | X | x |
| Child-Pugh score | X | X | X | X | X | X | X | X |
| calculation | | | | | | | | |
| Non-fasting | | X | | | | X ^{f} | X ^{f} | X |
| plasma/serum biomarkers | | | | | | | | |
| Fasting plasma | | X | | | | X ^{f} | X ^{f} | X |
| biomarkers | | | | | | | | |
| Spot urine: sodium, | X | X | X ^{f} | X ^{f} | X ^{f} | X ^{f} | X ^{f} | X |
| potassium, creatinine | | | | | | | | |
| Spot urine: urea, uric | | X | | | | X ^{f} | X ^{f} | X |
| acid, osmolality, glucose, | | | | | | | | |
| and albumin | | | | | | | | |
| HbA1c, full lipid profile ^{l} | | X | | | | | X ^{f} | X |
| Collect and store serum | | X | | | | X ^{f} | X ^{f} | |
| and plasma samples for | | | | | | | | |
| future exploratory | | | | | | | | |
| assessment of biomarkers | | | | | | | | |
| Collect and store urine | | X | | | | X ^{f} | X ^{f} | |
| samples for future | | | | | | | | |
| exploratory assessment of | | | | | | | | |
| biomarkers | | | | | | | | |
| Optional genetic | | | X | | | | | |
| sampling (blood) | | | | | | | | |

^{a}Visit can take place during up to 3 different days.
^{b} Participants have to be fasted for at least 8 hours prior to study intervention administration until the HVPG and VCTE/Fibroscan assessments have taken place. Participants will be allowed to drink water until 1 hour before and from 2 hours after study intervention administration and after completion of HVPG and VCTE/Fibroscan examinations. If the participant is on a non-selective beta-blocker to reduce HVPG, the morning dose should not be taken, but taken after the HVPG and Fibroscan assessments.
^{c} Brief physical examination.
^{d} Results need to be available before the baseline visit.
^{e} To be assessed ≥3 hours after study intervention administration. Note that randomisation should not take place before first HVPG recording at the study centre has been reviewed and concluded to be of good quality. If assessment of HVPG recording conclude that it is not of good enough quality, the study participant will not be randomised.
^{f} To be done pre-dose, unless otherwise stated.
^{g} PK sample at early termination visit only. If no dosing takes place, sample at any time during the study centre visit and document exact date and time.
^{h} Samples for PK profile collected over 5 hours at the study centre. Sampling times: predose, 0.5 to 1.0, 1.5 to 2.0, 2.5 to 3.0, 3.5 to 4.0, 4.5 to 5.5 hours postdose; exact clock times need to be documented.
ⁱ PK profile can be done at Visit 7 if not possible at Visit 6. At the day selected for the PK profile, the study intervention has to be administered at the study centre so that PK sampling according to schedule is possible.
^{j} Participants should abstain from using nicotine-containing products from the night prior to and during the visit.
^{k} If a SAE occurs within 48 hours after the last dose, a PK sample will be taken if possible. The exact time of last dosing and of blood sampling will be recorded.
^{l} HDL, total cholesterol and triglycerides.
^{m} Note that randomisation should not take place before first HVPG recording at the study centre has been reviewed by central reader.
ⁿ Study intervention intake will take place at the study centre, except at visits of HVPG assessment and VCTE/Fibroscan assessment when participants may take study intervention at home, visit procedures are separated on different days (but if a PK profile is performed at the visit, study intervention intake will take place at the study centre).
^{o} Pre-dose sample if dosing at the study centre; when the study intervention is taken at home, a post-dose sample may be taken at any time during the visit instead (record both exact dosing and sampling time).
^{p} VCTE/Fibroscan may take place on a separate day (Days 5 to 1) and assessed ≥3 hours after study intervention administration. VCTE/Fibroscan assessment of spleen stiffness should only be done if technically feasible to complete an accurate spleen stiffness measurement and is up to the discretion of the investigator at the study centre.
^{q} ECG assessment will be done during the HVPG procedure.
Abbreviations: AUDIT = Alcohol use Disorders Identification Test; BMI = body mass index; ECG = electrocardiogram; ECHO = echocardiogram; eGFR = estimated glomerular filtration rate; FSH = follicle-stimulating hormone; h = hour; HbA1c = haemoglobin A1c; HVPG = hepatic venous pressure gradient; IRT = Interactive Response Technology; MELD = model for end stage liver disease; MRI = magnetic resonance imaging; PK = pharmacokinetics; RTSM = Randomisation and Trial Supply Management; SAE = serious adverse event; VCTE = vibration-controlled transient elastography.

Table 2 shows Part B schedule of activities.

TABLE 2
Part B Schedule of Activities

| | | Treatment period | | | | | | | | Follow-up/early |
|---|---|---|---|---|---|---|---|---|---|---|
| Screening period | | Randomisation | Visits | | | | | | | termination |
| Visit number | | | | | | | | | | |
| 1 | 2 | 3 | 4 | 5 | 6 | 7 | 8 | 9 | 10 | 11 |
| Week | | | | | | | | | | |
| −6 | −1^{a} | 0^{a} | 1 | 2 | 3 | 6^{a} | 9 | 12 | 16^{a} | 18 |
| Study day | | | | | | | | | | |
| −35 | −5 | 1 | 8 | 15 | 22 | 43 | 64 | 85 | 113 | 127 |

Visit window (days)

| | | | | | | | | |
|---|---|---|---|---|---|---|---|---|
| ±4 | ±2 | ±2 | ±2 | ±5 | ±3 | ±3 | ±4 | ±3 |

| | | | | | |
|---|---|---|---|---|---|
| Informed consent | X | | | | |
| Inclusion/exclusion | X | X | X | | |
| criteria | | | | | |
| Alcohol breath test | X | | X | X | X |
| Screen for drugs of | X | | | | |
| abuse | | | | | |
| AUDIT | X | | | | |
| questionnaire | | | | | |
| Enrolment in | X | | | | |
| IRT/RTSM | | | | | |
| Randomization in | | | X | | |
| IRT/RTSM ^{o} | | | | | |

Routine clinical procedures

| | | | | | | | | | | | |
|---|---|---|---|---|---|---|---|---|---|---|---|
| Demography and | X | | | | | | | | | | |
| baseline | | | | | | | | | | | |
| characteristics | | | | | | | | | | | |
| (including smoking | | | | | | | | | | | |
| history and alcohol | | | | | | | | | | | |
| Medical/surgical | X | | | | | | | | | | |
| history | | | | | | | | | | | |
| Physical | X | | X | X | X ^{c} | X ^{c} | X ^{c} | X ^{c} | X ^{c} | X | X |
| examination | | | | | | | | | | | |
| Concomitant | X | | X | X | X | X | X | X | X | X | X |
| Medication | | | | | | | | | | | |
| Body weight | X | | X | X | X | X | X | X | X | X | X |
| Height, BMI | X | | | | | | | | | | |
| calculation | | | | | | | | | | | |
| ECG | X | X ^{s} | X (predose, | X | X | X | X ^{s} | X | X | X | X |
| | | | 2 hours postdose) | | | | | | | | |
| FSH (females | X ^{d} | | | | | | | | | | |
| only) | | | | | | | | | | | |
| Pregnancy tests | X | | X | | | | | | | | X |
| (females only; | (serum) | | | | | | | | | | |
| serum or urine) | | | | | | | | | | | |

Study intervention

| | | | | | | | | |
|---|---|---|---|---|---|---|---|---|
| Study intervention | X | | | X | X | X | X ^{k} | |
| dispensed | | | | | | | | |
| Study intervention | | X | X | X | X | X | X | X |
| accountability | | | | | | | | |
| (counting | | | | | | | | |
| remaining tablets | | | | | | | | |
| and capsules) | | | | | | | | |
| Study intervention | X | X | X | X | X | X | X | X |
| intake ^{p} | | | | | | | | |

Efficacy assessments

| HVPG ^{e} | | X ^{b,l} | | | | | X ^{b,l} | | | | |
|---|---|---|---|---|---|---|---|---|---|---|---|
| VCTE/Fibroscan r | | | X ^{b} | | | | X ^{b} | | | X ^{b} | |
| ECHO or cardiac | X (any | | | | | | | | | | |
| MRI ^{g} | time | | | | | | | | | | |
| | during | | | | | | | | | | |
| | screening | | | | | | | | | | |
| | period) | | | | | | | | | | |
| Daily digital body | | X | Daily home measurement using a digital device | | | | | | | | |
| weight | | | from baseline to end of follow-up. | | | | | | | | |
| measurement | | | | | | | | | | | |
| Bioimpedance | | X | X | X | X | X | X | X | X | X | X |
| spectroscopy | | | | | | | | | | | |

Safety assessments

| | | | | | | | | | | | |
|---|---|---|---|---|---|---|---|---|---|---|---|
| Adverse event | X | X | X | X | X | X | X | X | X | X | X |
| review ^{m} | SAEs | SAEs | | | | | | | | | |
| | only | only | | | | | | | | | |
| Pulse and | X | X | X (predose, | X | X | X | X | X | X | X | X |
| orthostatic test | | | 60, 120, 240 | | | | | | | | |
| (supine and | | | minutes) | | | | | | | | |
| standing blood | | | | | | | | | | | |
| pressure) | | | | | | | | | | | |
| Body temperature | X | X | X | X | X | X | X | X | X | X | X |
| Safety clinical | X | X | X ^{f} | X ^{f} | X ^{f} | X ^{f} | X ^{f} | X ^{f} | X ^{f} | X ^{f} | X |
| chemistry, | | | | | | | | | | | |
| electrolyte, | | | | | | | | | | | |
| coagulation and | | | | | | | | | | | |
| haematology | | | | | | | | | | | |
| HIV Serology | X | | | | | | | | | | |
| Other safety test: | X | X | X ^{f} | X ^{f} | X ^{f} | X ^{f} | X ^{f} | X ^{f} | X ^{f} | X ^{f} | X |
| Glutamate | | | | | | | | | | | |
| Dehydrogenase | | | | | | | | | | | |
| (GLDH), Serum | | | | | | | | | | | |
| Osmolality, BNP | | | | | | | | | | | |
| and NT-pro BNP | | | | | | | | | | | |

PK assessments

| | | | | | | | | |
|---|---|---|---|---|---|---|---|---|
| Pre-dose PK | X | X | X | X | X | X | X | X ^{h} |
| plasma sample ^{q} | | | | | | | | |
| PK profile | | | | | X ^{i,j} | | | |

Exploratory assessments

| | | | | | | | | | | | |
|---|---|---|---|---|---|---|---|---|---|---|---|
| MELD score | X | X | X | X | X | | X | X | X | X | X |
| calculation | | | | | | | | | | | |
| eGFR calculation | x | X | X | X | x | x | X | x | x | X | x |
| Child-Pugh score | X | X | X | X | X | X | X | X | X | X | X |
| calculation | | | | | | | | | | | |
| Non-fasting | | X | | | | X ^{f} | X ^{f} | | X ^{f} | X ^{f} | X |
| plasma/serum | | | | | | | | | | | |
| biomarkers | | | | | | | | | | | |
| Fasting plasma | | X | | | | X ^{f} | X ^{f} | | X ^{f} | X ^{f} | X |
| biomarkers | | | | | | | | | | | |
| Spot Urine: | X | X | X ^{f} | X ^{f} | X ^{f} | X ^{f} | X ^{f} | X ^{f} | X ^{f} | X ^{f} | X |
| sodium, potassium, | | | | | | | | | | | |
| creatinine | | | | | | | | | | | |
| Spot urine: urea, | | X | | | | X ^{f} | X ^{f} | | X ^{f} | X ^{f} | X |
| uric acid, | | | | | | | | | | | |
| osmolality, | | | | | | | | | | | |
| glucose, and | | | | | | | | | | | |
| albumin | | | | | | | | | | | |
| HbA1c, full lipid | | X | | | | | | | | X ^{f} | X |
| profile ^{,n} | | | | | | | | | | | |
| Collect and store | | X | | | | X ^{f} | X ^{f} | | X ^{f} | X ^{f} | |
| serum and plasma | | | | | | | | | | | |
| samples for future | | | | | | | | | | | |
| exploratory | | | | | | | | | | | |
| assessment of | | | | | | | | | | | |
| biomarkers | | | | | | | | | | | |
| Collect and store | | X | | | | X ^{f} | X ^{f} | | X ^{f} | X ^{f} | |
| urine samples for | | | | | | | | | | | |
| future exploratory | | | | | | | | | | | |
| assessment of | | | | | | | | | | | |
| biomarkers | | | | | | | | | | | |
| Optional genetic | | | X | | | | | | | | |
| sampling (blood) | | | | | | | | | | | |

^{a}Visit can take place during up to 3 different days.
^{b} Participants have to be fasted for at least 8 hours prior to study intervention administration until the HVPG and VCTE/Fibroscan assessments have taken place. Participants will be allowed to drink water until 1 hour before and from 2 hours after study intervention administration and after completion of HVPG and VCTE/Fibroscan examinations. If the participant is on a non-selective beta-blocker to reduce HVPG, the morning dose should not be taken, but taken after the HVPG and Fibroscan assessments.
^{c} Brief physical examination.
^{d} Results need to be available before the baseline visit.
^{e} To be assessed ≥3 hours after study intervention administration. Note that randomisation should not take place before first HVPG recording at the study centre has been reviewed and concluded to be of good quality. If assessment of HVPG recording conclude that it is not of good enough quality, the study participant will not be randomised.
^{f} To be done pre-dose, unless otherwise stated.
^{g} Only if historical data in the prior 12 months is not available.
^{h} PK sample at early termination visit only. If no dosing takes place, sample at any time during the study centre visit and document exact date and time.
ⁱ Samples for PK profile collected over 5 hours at the study centre. Sampling times: predose, 0.5 to 1.0, 1.5 to 2.0, 2.5 to 3.0, 3.5 to 4.0, 4.5 to 5.5 hours postdose; exact clock times need to be documented.
^{j} PK profile can be done at Visit 9 if not possible at Visit 8. At the day selected for the PK profile, the study intervention has to be administered at the study centre so that PK sampling according to schedule is possible.
^{k} Two bottles of study intervention to be dispensed.
^{l} Participants should abstain from using nicotine-containing products from the night prior to and during the visit.
^{m} If a SAE occurs within 48 hours after the last dose, a PK sample will be taken if possible. The exact time of last dosing and of blood sampling will be recorded.
ⁿ HDL, total cholesterol and triglycerides.
^{o} Note that randomisation should not take place before first HVPG recording at the study centre has been reviewed by central reader.
^{p} Study intervention intake will take place at the study centre, except at visits of HVPG assessment (Visit 7) and VCTE/Fibroscan assessment (Visits 7 and 10) when participants may take study intervention at home, visit procedures are separated on different days (but if a PK profile is performed at the visit, study intervention intake will take place at the study centre).
^{q} Pre-dose sample if dosing at the study centre; when the study intervention is taken at home, a post-dose sample may be taken at any time during the visit instead (record both exact dosing and sampling time).
^{r} VCTE/Fibroscan may take place on a separate day (Days 5 to 1) and assessed ≥3 hours after study intervention administration. VCTE/Fibroscan assessment of spleen stiffness should only be done if technically feasible to complete an accurate spleen stiffness measurement and is up to the discretion of the investigator at the study centre.
^{s} ECG assessment will be done during the HVPG procedure.
Abbreviations: AUDIT = Alcohol use Disorders Identification Test; BMI = body mass index; ECG = electrocardiogram; ECHO = echocardiogram; eGFR = estimated glomerular filtration rate; FSH = follicle-stimulating hormone; h = hour; HbA1c = haemoglobin A1c; HVPG = hepatic venous pressure gradient; IRT = Interactive Response Technology; MELD = model for end stage liver disease; MRI = magnetic resonance imaging; PK = pharmacokinetics; RTSM = Randomisation and Trial Supply Management; SAE = serious adverse event; VCTE = vibration-controlled transient elastography.

### 1.2 Participants.

In Part A, approximately 30 participants having good quality baseline HVPG are randomized to 2 treatment groups of 15 participants per treatment group. With an estimated 20% premature treatment discontinuation, this allows for 12 evaluable participants per treatment group with evaluable HVPG measurements at baseline and Week 6 (primary endpoint).

In Part B, approximately 110 participants having good quality baseline HVPG are randomized to 5 treatment groups of 22 participants per treatment group. With an estimated 20% premature treatment discontinuation, this allows for 18 evaluable participants per treatment group with evaluable HVPG measurements at baseline and Week 6 (primary endpoint).

### 1.3 Intervention Groups and Duration.

In Part A, participants who meet the eligibility criteria and agree to participate are randomized to one of the following 2 treatment groups (15 participants per group):

Treatment Group 1: placebo matching zibotentan capsule+placebo matching dapagliflozin tablet.

Treatment Group 2: zibotentan capsule 2.5 mg+dapagliflozin tablet 10 mg.

The study intervention is a once daily dose of the assigned study intervention (oral capsules and tablets) for 6 weeks, in addition to their standard of care therapy. Total study duration for participants in Part A is approximately 14 weeks (including the screening and follow up period).

In Part B, participants who meet the eligibility criteria and agree to participate are randomized to one of the following 5 treatment groups (22 participants per group):

Treatment Group 1: placebo matching zibotentan capsule+placebo matching dapagliflozin tablet.

Treatment Group 2: placebo matching zibotentan capsule+dapagliflozin tablet 10 mg.

Treatment Group 3: zibotentan capsule 1 mg+dapagliflozin tablet 10 mg.

Treatment Group 4: zibotentan capsule 2.5 mg+dapagliflozin tablet 10 mg.

Treatment Group 5: zibotentan capsule 5 mg+dapagliflozin tablet 10 mg.

A staggered randomization scheme is applied in Part B. Eligible participants are randomized to treatment groups 1-4 with equal allocation. targeting 22 participants per treatment group (approximately 88 participants in total).

In some embodiments, participants in Part B are stratified by the presence of clinically detectable (grade 2) ascites at the time of randomization to ensure an approximate balance between treatment groups within each sub population.

The study intervention is a once daily dose of the assigned study intervention (oral capsules and tablets) for 16 weeks, in addition to their standard of care therapy. Total study duration for participants in Part B is approximately 24 weeks (including the screening and follow up period).

Eligibility Criteria of participants in Part A:
(a) Clinical and/or histological diagnosis of cirrhosis with either (i) features of portal hypertension or (ii) liver stiffness >21 kPa.
(b) MELD score <15.
(c) Child-Pugh score ≤6.
(d) No clinically evident ascites
(e) No evidence of worsening of hepatic function (e.g., no clinically significant change in signs, symptoms, or laboratory parameters of hepatic disease status) within the last month prior to dosing, as determined by the investigator or usual practitioner.
(f) HVPG recording of good enough quality as judged by a central reader.

Eligibility Criteria of participants in Part B:
(a) Clinical and/or histological diagnosis of cirrhosis with features of portal hypertension.
(b) MELD score <15.
(c) Child-Pugh score <10.
(d) No ascites or ascites up to grade 2 without change in diuretic treatment within the last month prior to first dose and no paracentesis within the last month or planned paracentesis in the next 4 months at screening.
(e) No evidence of worsening of hepatic function (e.g., no clinically significant change in signs, symptoms, or laboratory parameters of hepatic disease status) within the last month prior to dosing, as determined by the investigator or usual practitioner.
(f) HVPG recording of good enough quality as judged by a central reader.

### Exclusion Criteria

Participants are excluded from the study if any of the following criteria apply:

### Medical Conditions (All Participants)

1. Any evidence of a clinically significant disease which in the investigator's opinion makes it undesirable for the participant to participate in the study.
2. Liver cirrhosis caused by chronic cholestatic liver disease.
3. ALT or AST ≥150 U/L and/or total bilirubin ≥3×ULN.
4. Any history of hepatocellular carcinoma.
5. Any history of portal venous thrombosis.
6. Liver transplant or expected liver transplantation within 6 months of screening.
7. History of TIPS or a planned TIPS within 6 months from enrolment into the study.
8. Positive alcohol breath test or screen for drugs of abuse (excluding drugs prescribed by the participants' usual physician) at screening.
9. Ongoing or history of significant use of alcohol expected to preclude correct adherence to study procedures.
10. Active treatment for HCV within the last 1 year or HBV antiviral therapy for less than 1 year.
11. Active urinary tract infection or genital infection
12. Uncontrolled diabetes mellitus (HbA1c >8% or >64 mmol/mol within the last month).
13. Participants with T1DM.
14. Renal transplant or chronic renal replacement therapy or short-term dialysis within the previous 6 months.
15. eGFR <60 mL/min/1.73m² (eGFRcr[AS]).
16. Acute coronary syndrome events within 3 months prior to screening.
17. Orthostatic hypotension or hypotension (systolic blood pressure <95 mmHg or diastolic blood pressure <60 mmHg).
18. Participants treated with strong CYP3A4 inhibitor or strong or moderate CYP3A4 inducer within 14 days (St. John's Wort 21 days) of study intervention administration (Error! Reference source not found.); this includes grapefruit and grapefruit juice, if consumed more often than occasionally, or, in larger quantities.
19. History or ongoing allergy/hypersensitivity, as judged by the investigator, to SGLT2i (e.g., dapagliflozin, empagliflozin), zibotentan, or drugs with a similar chemical structure to zibotentan.
20. Any clinically significant chronic disease or disorder (e.g., cardiovascular, gastrointestinal, liver, renal, neurological, musculoskeletal, endocrine, metabolic, psychiatric, major physical impairment) which, as judged by the investigator, might put the participant at risk because of participation in the study, or probable alternative primary reason for participant's symptoms in judgment of investigator, for example:
   a. Anaemia defined as haemoglobin (Hb) levels <100 g/L or 10 g/dL.
   b. Wernicke's encephalopathy or Korsakoff's syndrome.
   c. History of HIV.
21. Any of the following regarding COVID-19:
   a. Symptoms of COVID-19 infection or a recent positive test in the 14 days prior to enrolment in the study.
   b. Participants hospitalised with COVID-19 infection within the last 3 months who required in-hospital medical care (oxygen therapy, mechanical ventilation, intensive care unit admission, etc.).
22. Acute liver injury caused by drug toxicity or by an infection.

### Medical Conditions (Part A Only)

1. INR >1.5.
2. Serum/plasma levels of albumin ≤35 g/L.
3. Platelet count <75×10⁹/L.
4. History of ascites
5. History of hepatic hydrothorax
6. History of portopulmonary syndrome
7. History of hepatic encephalopathy
8. History of variceal haemorrhage
9. History of acute kidney injury
10. History of heart failure, including high output heart failure (e.g., due to hyperthyroidism or Paget's disease)

### Medical Conditions (Part B Only)

1. INR >1.7.
2. Serum/plasma levels of albumin ≤28 g/L.
3. Platelet count <50×/10⁹L.
4. Acute kidney injury within 3 months of screening.
5. History of encephalopathy of West Haven grade 2 or higher.
6. History of variceal haemorrhage within 6 months prior to screening.
7. NYHA functional heart failure class III or IV or with unstable heart failure requiring hospitalisation for optimisation of heart failure treatment and who are not yet stable on heart failure therapy within 6 months prior to screening.
8. Heart failure due to cardiomyopathies that would primarily require specific other treatment: e.g., cardiomyopathy due to pericardial disease, amyloidosis or other infiltrative diseases, cardiomyopathy related to congenital heart disease, primary hypertrophic cardiomyopathy, cardiomyopathy related to toxic or infective conditions (i.e. chemotherapy, infective myocarditis, septic cardiomyopathy).
9. High output heart failure (e.g., due to hyperthyroidism or Paget's disease).
10. Heart failure due to primary cardiac valvular disease/dysfunction, severe functional mitral or tricuspid valve insufficiency, or planned cardiac valve repair/replacement

### 1.4 Statistical Methods.

For Part A, the primary efficacy endpoint is the absolute change in HVPG at Week 6, and the change is analyzed using ANCOVA with baseline values taken as a covariate for the comparison between the zibotentan/dapagliflozin group and the placebo group. In some embodiments, there is a difference of 2 mmHg or more in mean absolute change in HVPG at Week 6 for the zibotentan/dapagliflozin group versus the placebo group, and a common standard deviation (SD) for difference in HVPG is 2.5 mmHg (Bai et al 2021) or less. Under this situation, 12 HVPG evaluable participants per group provides 80% power to detect the difference at Week 6 with a type I error of 15% (1-sided). With an estimated 20% premature treatment discontinuation, Part A randomizes a total of 30 participants (15 per group).

For Part B, the primary efficacy endpoint is HVPG response, where a responder is defined as a ≥20% decrease in HVPG or a reduction to or below 12 mmHg in HVPG from baseline at Week 6. The HVPG response at Week 6 is analyzed via Cochran Mantel Haenszel test accounting for the stratification factors. Pairwise comparison is made for zibotentan and dapagliflozin in combination versus placebo. With one-sided type I error of 5%, 18 HVPG evaluable participants per group in Part B provides 80% power to detect the difference at Week 6 between a placebo HVPG response rate of 9% and a zibotentan/dapagliflozin response rate of 45% assuming zibotentan/dapagliflozin effect is similar to unselective beta blockers. With an estimated 20% premature treatment discontinuation, Part B randomizes 22 participants in each of the 5 treatment groups to have 90 participants (18 participants per treatment group) with evaluable HVPG measurements at baseline and Week 6. Up to 110 participants are therefore randomized in this study.

The change in body weight, body water volumes, body fat mass, accumulated dosage of loop-diuretic equivalents, and blood pressure in Part A and Part B are analyzed using mixed model repeated measures (MMRM) methodology. The analytic model includes the fixed categorical effects of stratification factor, treatment, visit, and treatment-by-visit interaction and continuous covariates of the baseline measurements. In some embodiments, this model also includes cohort (by randomization) as a covariate in the case of randomization adjustment. An unstructured covariance structure is used for the within-participant errors.

In the previous Phase III program in oncology, zibotentan 10 mg was used and extensive safety data are available for this dose. Based on the zibotentan hepatic impairment study (Tomkinson et al, 2011), exposure is expected to be 1.45 times higher in participants with moderate hepatic impairment compared to healthy control participants. Example 1 includes participants with mild or moderate hepatic impairment and with normal renal function or mild renal impairment (MELD score <15 and eGFR >60 mL/min/1.73m2) and dosing is adjusted as described below.

Part A is designed to give proof of concept. Participants with less severe disease, defined as Child-Pugh A and no history of decompensation events, receive 2.5 mg zibotentan in combination with 10 mg dapagliflozin or placebo. After the combination therapy is determined to be safe in these participants, then Part B commences which also includes participants with Child-Pugh B, a history of decompensation events, or ascites. In Part B, initially only two dose levels of 1 mg and 2.5 mg zibotentan are studied in combination with dapagliflozin, conservatively representing 1/10 and ¼ of the zibotentan dose in the previous oncology program. After the two lower dose levels for safety and tolerability have been evaluated, a 5 mg zibotentan cohort in combination with 10 mg of dapagliflozin is enrolled. This is intended to increase the dose range to 5-fold and evaluate the potential benefits of a 5 mg dose while safeguarding subjects enrolled in the study with the staggered design.

Dapagliflozin 10 mg has been extensively studied as monotherapy. A higher exposure of dapagliflozin is expected in participants with renal and/or hepatic impairment compared to healthy participants but does not require dose adjustment given the safety and tolerability profile of dapagliflozin. Dapagliflozin 10 mg once daily did not lead to any unique safety or tolerability signals in participants with renal impairment in both chronic kidney disease (≥25 mL/min/1.73 m2; DAPA-CKD outcomes study) and heart failure populations (DAPA HF outcomes study). Doses of dapagliflozin up to 100 mg for 14 days to healthy volunteers have been shown to be tolerable. Dapagliflozin 10 mg once daily has therefore also been selected for the planned clinical program in cirrhosis with features of portal hypertension (Dapagliflozin Investigator's Brochure).

### 2. Study Interventions Administered

### 2.1 Study Intervention.

As shown in Table 3 below, all participants receive 1 capsule (zibotentan or matching placebo) and 1 tablet (dapagliflozin or matching placebo) at every dosing.

TABLE 3

| | Part A | | | | |
|---|---|---|---|---|---|
| | Treatment | | | Treatment | |
| | group 1 | NA | NA | group 2 | NA |
| | Part B | | | | |
| | Treatment | Treatment | Treatment | Treatment | Treatment |
| | group 1 | group 2 | group 3 | group 4 | group 5 |
| Intervention | Placebo | dapagliflozin | zibotentan + | zibotentan + | zibotentan + |
| name | | | dapagliflozin | dapagliflozin | dapagliflozin |
| Type | Placebo | Drug/placebo | Drug | Drug | Drug |
| Dose | placebo capsule | placebo capsule | zibotentan | zibotentan | zibotentan |
| formulation | (matching | (matching | capsule | capsule | capsule |
| | zibotentan | zibotentan | dapagliflozin | dapagliflozin | dapagliflozin |
| | capsule) | capsule) | tablet | tablet | tablet |
| | placebo tablet | dapagliflozin | | | |
| | (matching | tablet | | | |
| | dapagliflozin | | | | |
| | tablet) | | | | |
| Dose | NA | 10 mg: | 1 mg: | 2.5 mg: | 5 mg: |
| levels | | dapagliflozin | zibotentan | zibotentan | zibotentan |
| | | | 10 mg: | 10 mg: | 10 mg: |
| | | | dapagliflozin | dapagliflozin | dapagliflozin |
| Route of | Oral | Oral | Oral | Oral | Oral |
| administration | | | | | |
| Use | Experimental | Experimental | Experimental | Experimental | Experimental |
| IMP and NIMP | IMP | IMP | IMP | IMP | IMP |

| | |
|---|---|
| Sourcing | zibotentan, dapagliflozin, and their respective matching placebo treatments will be |
| | supplied centrally through AstraZeneca. |
| Packaging and | For each strength, zibotentan and matching placebo will be supplied in HDPE bottles. |
| labelling | Dapagliflozin and matching placebo will be supplied in HDPE bottles. |
| | All bottles will be labelled as per country requirement. |

GMP = Good Manufacturing Practice; HDPE = high density polyethylene; IMP = investigational medicinal product; NA = not applicable; NIMP = non investigational medicinal product.

### 3. Assessments

### 3.1 Efficacy Assessments

### 3.1.1 Primary Variable: Hepatic Venous Pressure Gradient

HVPG assessments are performed at timepoints specified in Table 1 and Table 2 At the day of the HVPG assessment, participants have to be fasted and avoid nicotine containing products until the HVPG assessment has taken place. If the participant is on a non-selective beta-blocker, the dose should not be taken in the morning before HVPG, but right after the HVPG assessment. HVPG assessment must not take place earlier than 3 hours after administration of study intervention. Therefore, the timing of HVPG assessment and intake of study intervention at Visit 7 is carefully planned. The baseline recordings are reviewed and found to be of good enough quality by the central reader before the participant can be randomized. A good enough HVPG recording is the result of a recording that adheres to all the requirements of the experiment. An acceptable tracing that can be used to derive HVPG data although not totally satisfactory, still allows to derive the more important information from several (even if not 3) measurements of WHVP, FHVP and HVPG, with consistent results even if the tracings have artefacts, or if some ancillary measurement is lacking (i.e., right atrial pressure, inferior vena cava pressure).

A balloon catheter is introduced via the transjugular approach into the hepatic vein under ultrasound/fluoroscopic guidance. Light sedation for participant comfort is provided. Midazolam is the preferred medication for sedation since it does not influence HVPG. Note that zibotentan leads to a 20% increase in midazolam exposure which is, however, unlikely to be clinically relevant with standard midazolam dosing. Once the catheter is in situ in the hepatic vein, an initial free venous pressure measurement is undertaken, followed by a wedge pressure measurement. HVPG is calculated from these two values. Measurements of HVPG are performed in triplicate.

### 3.1.2 Secondary Variables

### 3.1.2.1 Electronic Scale Measurement of Body Weight.

Body weight is measured at study visits. In addition, participants measure their body weight every morning using a digital device provided.

### 3.1.2.2 Bioimpedance Spectroscopy.

Bioimpedance spectroscopy (BIS) is performed at the timepoints specified in the SoA (Table 1 and Table 2) to monitor body fluid volumes. This non-invasive procedure uses skin electrodes to pass a low-level alternating current through the body and measures the impedance to the flow of this current. Tissues such as fat and bone act as insulators; whereas electrolyte body fluids conduct electrical current and as the fluid increases, impedance to current flow decreases (i.e., changes in impedance are inversely proportional to the volume of the extracellular fluid in the body). At low frequencies, cell membranes are non-conductive and current passes only through the extracellular fluid, while at high frequencies, the current passes through cell membranes in addition to the extra-and intracellular fluids.

### 3.1.3 Exploratory Variables

### 3.1.3.1 Vibration Controlled Transient Elastography (VCTE).

VCTE is performed at the timepoints specified in Table 1 and Table 2 in the fasting state as well as ≥3 hours after study intervention administration. If the participant is on a non-selective beta-blocker, the dose isn't taken in the morning before VCTE, but right after the VCTE assessment. Fibroscan equipment that allow measurement of spleen stiffness is used. Spleen stiffness is collected as an exploratory endpoint to investigate to what extent it may be used as a surrogate for changes in hepatic portal venous pressure gradient. The measurement is preferably done by the same examiner at all occasions.

VCTE/Fibroscan assessment of spleen stiffness is only done if technically feasible to complete an accurate spleen stiffness measurement.

3.1.3.2 Serum Analysis of Creatinine and Cystatin C (eGRF).

The participant's eGFR is calculated according to the 2021 CKD EPI equation, based on serum creatinine concentration alone (Inker et al, 2021) for eligibility and safety, referred to below as eGFR1. Additionally, eGFR for exploratory assessment is calculated based on both serum creatinine and serum cystatin C concentrations (Inker et al, 2021), referred to below as eGFR2.

Estimated GFR using serum creatinine alone or together with serum cystatin C is calculated as follows:

eGFR1=142×min(Scr/κ,1)^{-0.241 (if female)/0.302} (if male)×max(Scr/κ,1)^{-1.200}×0.994^{Age}×1.012 [if female]

eGFR2=135×min(Scr/κ,1)^{-0.219 (if female)/0.144 (if male)}×max(Scr/κ,1)^{-0.544}×min(Scys/0.8, 1)^{-0.323}×max(Scys/0.8,1)^{-0.778}×0.996^{Age}×0.963 [if female]

where:
S_{cr}=serum creatinine (mg/dL)
S_{cys}=serum cystatin C (mg/L)
κ=0.7 for females; and 0.9 for males
min=minimum of Scr/κ or 1
max=maximum of Scr/κ or 1

### 3.1.3.3 Laboratory Assessments for Exploratory Analysis

Blood and urine samples are collected as shown in the SoA (Table 1 and Table 2) to measure levels of the following biomarkers for analysis of exploratory variables:
Plasma/serum AST, ALT, FIB 4 are derived from safety variables (Part B only).
MELD score (MELD=3.78×ln[serum bilirubin (mg/dL)]+11.2×ln[INR]+9.57×ln[serum creatinine (mg/dL)]+6.43) are calculated at every visit.
Plasma/serum potassium, sodium, creatinine, cystatin C, uric acid, serum osmolality, BUN, BNP, NTproBNP, HbA1c, hematocrit, hemoglobin, ET1, ELDP, CTproET1, and copeptin are non-fasting plasma biomarkers in the SoA.
Fasting plasma/serum glucose, fasting insulin, fasting beta hydroxybutyrate are fasting plasma biomarkers in the SoA. Total cholesterol, triglycerides and HDL-cholesterol are additional fasting plasma biomarkers in the SoA.
Spot urine test for potassium, sodium, and creatinine, and for urea, uric acid, osmolality, glucose, and albumin, are measured at visits specified in the SoA.
eGFR are calculated at every visit.

The Child-Plugh scores are calculated as found in Table 5:

TABLE 5
Child Pugh Score

| | Points | | |
|---|---|---|---|
| Clinical and laboratory criteria | 1 | 2 | 3 |
| Encephalopathy | None | Mild to moderate | Severe (West Haven |
| | | (West Haven grade 1 | grade 3 or 4) |
| | | or 2) | |
| Ascites | None | Mild to moderate | Severe (diuretic |
| | | (diuretic responsive) | refractory) |
| Bilirubin (mg/dL) | <2 | 2-3 | >3 |
| Albumin (g/dL) | >3.5 | 2.8-3.5 | <2.8 |
| Prothrombin time | | | |
| Seconds prolonged | <4 | 4-3 | >6 |
| International normalised ratio | <1.7 | 1.7-2.3 | >2.3 |

Child-Turcotte-Pugh class obtained by adding score for each parameter (total points)
Class A = 5 to 6 points (least severe liver disease)
Class B = 7 to 9 points (moderately severe liver disease)
Class C = 10 to 15 points (most severe liver disease)

### 3.2.1 Vital Signs

Vital signs are collected at timelines as specified in the SoA (Table 1 and Table 2). Vital signs include supine and standing blood pressure, pulse, and body temperature.

Routine blood pressure, pulse, and body temperature are assessed as outlined in the SoA (Table 1 and Table 2), prior to blood collection for laboratory tests with the participant resting in a supine position using a completely automated device. Manual techniques are used only if an automated device is not available.

All participants are also equipped with home blood pressure monitor, to be used if the participant suffers from symptoms suggestive of low blood pressure or orthostatic hypotension.

Vital sign measurements in a supine position are preceded by at least 5 minutes of rest for the participant in a quiet setting without distractions (e.g., television, cell phones) and are consist of 1 pulse, 1 body temperature, and 3 blood pressure measurements (3 consecutive blood pressure readings are recorded at intervals of at least 1 minute). The average of the 3 blood pressure readings is recorded on the eCRF. In addition, a standing blood pressure measurement should be recorded after 2 to 5 minutes in the standing position. These assessments are preferably done in the morning. If orthostatic hypotension is confirmed during the test, it is reported as an AESI including symptoms related to the measurement of orthostatic vitals if present.

There is real-time safety monitoring of body weight online data from the home based monitoring devices by the Study Physician. The Study Physician would alert the respective investigator if any significant weight gain (>2 kg per week) is seen.

### 3.2.2 Electrocardiograms

Single 12-lead ECGs are performed after the participant has been resting in a supine position for at least 10 minutes, at the visits outlined in the SoA (Table 1 and Table 2).

Throughout the study, clinically relevant new findings or worsening of a pre-existing finding in the ECGs (parameters or abnormal findings in the tracing) is considered an AE and is recorded in the AE CRF form.

### 3.2.3 Clinical Safety Laboratory Assessments

Blood and urine samples for determination of clinical chemistry, haematology, and urinalysis for safety analysis are taken at the visits indicated in Table 1 and Table 2. Laboratory assessments for exploratory analysis are presented above.

The following laboratory safety variables are measured as found in Table 5.

TABLE 5
Laboratory Safety Variables
Haematology and differential panel

| | |
|---|---|
| White blood cell (WBC) count | Lymphocytes absolute count |
| Red blood cell (RBC) count | Monocytes absolute count |
| Haemoglobin (Hb) | Eosinophils absolute count |
| Haematocrit (HCT) | Basophils absolute count |
| Neutrophils absolute count | Platelet absolute count |

Chemistry panel

| | |
|---|---|
| BUN | Alanine aminotransferase (ALT) |
| Serum creatinine and calculated eGFR | Aspartate aminotransferase (AST) |
| (CKD-EPI) | |
| Total bilirubin and direct bilirubin | Alkaline phosphatase (ALP) |
| Albumin | Creatine kinase (CK) |
| Calcium | Glucose |
| Phosphate | Gamma-glutamyl transferase (GGT) |

Other

| | |
|---|---|
| Glutamate Dehydrogenase (GLDH) | Serum Osmolality |

NT-pro BNP and BNP

Coagulation groups (frozen samples)

| | |
|---|---|
| International normalised ratio (INR) | Prothrombin time (PT) |

Partial thromboplastin time (APTT)

Electrolyte panel

| | |
|---|---|
| Bicarbonate (HCO₃−) | Sodium |
| Chloride (Cl−) | Potassium |
| Magnesium (Mg⁺) | |

Urine screen for drugs of abuse

| | |
|---|---|
| Amphetamine/MDMA | Opiates |
| Antidepressants | Methadone |
| Barbiturates | Phencyclidine (PCP) |
| Benzodiazepines | Propoxyphene |
| Cannabinoids | Cocaine |

HIV serology

Human Immunodeficiency Virus (HIV) load

### 3.2 Pharmacokinetics

Plasma samples are collected for measurement of concentrations of zibotentan and dapagliflozin as specified in the SoA (Table 1 and Table 2).

Samples may be collected at additional time points during the study if warranted and agreed upon between the investigator and the sponsor, e.g., for safety reasons such as an SAE. The timing of sampling may be altered during the course of the study (e.g., to obtain data closer to the time of peak or trough matrix concentrations) to ensure appropriate monitoring.

Samples for determination of drug concentration in plasma are assayed using an appropriately validated bioanalytical method. Incurred sample reproducibility analysis, if any, would be performed alongside the bioanalysis of the test samples.

### 3.3 Pharmacodynamics

Plasma/serum parameters are measured during the study for the evaluation of the exploratory endpoints. Parameters are measured from the blood samples collected for safety analyzed at a central core laboratory. Blood samples are collected as described in the SoA (Table 1 and Table 2) and analyzed by a central laboratory.

### 3.4 Study Endpoints

Study endpoints are summarized in Table 4 and Table 5. The primary efficacy objective for Part A is the absolute change in HVPG at 6 weeks from baseline in participants treated with 2.5 mg zibotentan combined with 10 mg dapagliflozin versus placebo. The primary efficacy objective for Part B is the proportion of participants treated with 1, 2.5, or 5 mg zibotentan combined with 10 mg dapagliflozin and 10 mg dapagliflozin monotherapy versus placebo achieving a ≥20% decrease in HVPG or a reduction to or below 12 mmHg in HVPG. Secondary objectives in both Parts A and B include the percentage change in HVPG and assessment of body weight, body fat mass, and body water volumes, accumulated dosage of loop diuretic equivalents, and blood pressure.

TABLE 4
Objectives and endpoints of Part A.

| | |
|---|---|
| Objectives | Endpoints |

Primary

| | |
|---|---|
| To evaluate the change from baseline in | Absolute change in HVPG from baseline to |
| HVPG on zibotentan and dapagliflozin in | Week 6. |
| combination versus placebo. | |

Secondary

| | |
|---|---|
| To evaluate the change from baseline in | Percent change in HVPG from baseline to |
| HVPG on zibotentan and dapagliflozin in | Week 6. |
| combination versus placebo. | |
| To evaluate the proportion of participants | HVPG response, where a responder is |
| achieving HVPG < 10 mmHg or a reduction | defined as HVPG < 10 mmHg or a reduction |
| in HVPG of ≥ 1.5 mmHg on zibotentan and | in HVPG of ≥ 1.5 mmHg from baseline to |
| dapagliflozin versus placebo. | Week 6. |
| To evaluate the effect of zibotentan and | Evaluation of change in body weight (kg) |
| dapagliflozin in combination versus placebo | over time course of study. |
| on change in body weight. | Percentage and absolute change from |
| | baseline in body weight at Week 6. |
| To evaluate the effect of zibotentan and | Percentage and absolute change in |
| dapagliflozin in combination versus placebo | accumulated dosage of loop-diuretic |
| on accumulated additional loop-diuretic | equivalents use from baseline to Week 6. |
| equivalents use. | |
| To evaluate the effect of zibotentan and | Change in total body water, extracellular |
| dapagliflozin in combination versus placebo | water and intracellular water volumes |
| on body water volumes and body fat mass. | from baseline to Week 6. |
| | Change in total body fat mass from baseline |
| | to Week 6. |
| To evaluate the effect of zibotentan and | Change in systolic and diastolic blood |
| dapagliflozin in combination versus placebo | pressure from baseline to Week 6. |
| on changes in office-based systolic and | |
| diastolic blood pressure. | |

Safety

| | |
|---|---|
| To assess the safety and tolerability of | AEs, SAEs, and DAEs. |
| zibotentan and dapagliflozin in combination | AESIs (new or worsening HF; other |
| versus placebo. | signs of fluid retention; orthostatic |
| | hypotension; UTI; GI; AKI). |
| | Vital signs. |
| | Clinical laboratory tests. |
| | ECG assessments. |

Exploratory

| | |
|---|---|
| To evaluate the effect of zibotentan and | Percentage and absolute change in spleen |
| dapagliflozin in combination versus placebo | stiffness from baseline to Week 6. |
| on spleen stiffness as measured with | |
| VCTE/Fibroscan^{t}. | |
| To assess the plasma exposure of zibotentan | Plasma concentrations of zibotentan and |
| and dapagliflozin in the participant | dapagliflozin per treatment, visit and |
| population. | timepoint. |
| To determine the effect of zibotentan and | Change in eGFR from baseline to Week 1 |
| dapagliflozin in combination versus placebo | and Week 6. |
| on eGFR. | |
| To assess the effect of zibotentan and | Evaluation of changes in blood and urine |
| dapagliflozin in combination versus placebo | biomarkers across time course of the study. |
| on plasma/serum potassium, sodium, | |
| creatinine, cystatin C, uric acid, plasma | |
| osmolality, BUN, BNP, fasting plasma | |
| glucose, fasting insulin, fasting | |
| beta-hydroxybutyrate, HbA1c, haematocrit, | |
| haemoglobin, ET-1, ELDurineP, | |
| CTproET-1 and copeptin levels, and on | |
| urine sodium, potassium, urea, uric acid, | |
| glucose, and albumin creatinine ratios as | |
| well as urine osmolality. | |
| To collect and store plasma, serum and | Evaluation of changes in blood and urine |
| urine samples for potential exploratory | biomarkers across time course of the study. |
| research aimed at exploring biomarkers | |
| involved in PK, PD, safety and tolerability | |
| related to zibotentan and dapagliflozin in | |
| combination versus placebo or related to | |
| liver diseases. | |
| Collect and store blood samples for genetic | Exploratory research into genes/genetic |
| research (according to each country's local | variation that may influence response to |
| and ethical procedures) (optional). | treatment. |

a. Home-based balance.
b. Office balance.
c. 40 mg furosemide = 1 mg bumetanide = 20 mg torsemide = 50 mg ethacrynic acid. Abbreviations: AE = adverse event; AESI = adverse event of special interest; AKI = acute kidney injury; DAE = discontinuation due to adverse event; ECG = electrocardiogram; GI = genital infection; HVPG = hepatic venous pressure gradient; SAE = serious adverse event; UTI = urinary tract infection.
d. VCTE/Fibroscan assessment of spleen stiffness should only be done if technically feasible to complete an accurate spleen stiffness measurement and is up to the discretion of the investigator at the study centre.
Abbreviations: AE = adverse event; AESI = adverse event of special interest; AKI = acute kidney injury; BNP = B-type natriuretic peptide; BUN = blood urea nitrogen; CTproET-1 = C-terminal pro-endothelin-1; DAE = discontinuation due to adverse event; ECG = electrocardiogram; eGFR = estimated glomerular filtration rate; ELDP = endothelin-like domain peptide; ET-1 = endothelin-1; FIB-4 = fibrosis-4; GI = genital infection; HbA1c = haemoglobin A1c; HE = hepatic encephalopathy; HVPG = hepatic venous pressure gradient; PD = pharmacodynamic; PK = pharmacokinetic; SAE = serious adverse event; UTI = urinary tract infection; VCTE = vibration-controlled transient elastography.

TABLE 5
Objectives and endpoints of Part B.

| | |
|---|---|
| Objectives | Endpoints |

Primary

| | |
|---|---|
| To evaluate the proportion of participants | HVPG response, where a responder is |
| achieving at least 20% decrease in HVPG or | defined as at least 20% decrease or a |
| a reduction to or below 12 mmHg in HVPG | reduction to or below 12 mmHg in HVPG |
| on zibotentan and dapagliflozin in | from baseline to Week 6. |
| combination and dapagliflozin monotherapy | |
| versus placebo. | |

Secondary

| | |
|---|---|
| To evaluate the change from baseline in | Percentage and absolute change in HVPG |
| HVPG on zibotentan and dapagliflozin in | from baseline to Week 6. |
| combination and dapagliflozin monotherapy | |
| versus placebo. | |
| To evaluate the effect of zibotentan and | Evaluation of change in body weight (kg) |
| dapagliflozin in combination and | over time course of study. |
| dapagliflozin monotherapy versus placebo | Percentage and absolute change from |
| on change in body weight. | baseline in body weight at Week 6 and |
| | Week 16. |
| To evaluate the effect of zibotentan and | Percentage and absolute change in |
| dapagliflozin in combination and | accumulated dosage of loop-diuretic |
| dapagliflozin monotherapy versus placebo | equivalents use from baseline to Week 6 and |
| on accumulated additional loop-diuretic | Week 16. |
| equivalents use. | |
| To evaluate the effect of zibotentan and | Change in total body water, extracellular |
| dapagliflozin in combination and | water and intracellular water volumes |
| dapagliflozin monotherapy versus placebo | from baseline to Week 6 and Week 16. |
| on body water volumes and body fat mass. | Change in total body fat mass from baseline |
| | to Week 6 and Week 16. |
| To evaluate the effect of zibotentan and | Change in systolic and diastolic blood |
| dapagliflozin in combination and | pressure from baseline to Week 6 and |
| dapagliflozin monotherapy versus placebo | Week 16. |
| on changes in office-based systolic and | |
| diastolic blood pressure. | |

Safety

| | |
|---|---|
| To assess the safety and tolerability of | AEs, SAEs, and DAEs. |
| zibotentan and dapagliflozin in combination | AESIs (new or worsening HF; other |
| and dapagliflozin monotherapy versus | signs of fluid retention; orthostatic |
| placebo. | hypotension; UTI; GI; AKI). |
| | Vital signs. |
| | Clinical laboratory tests. |
| | ECG assessments. |

Exploratory

| | |
|---|---|
| To evaluate the proportion of participants | HVPG response, where a responder is |
| who exhibit 10%, 20%, and 30% decrease | defined as a 10%, 20% and 30% decrease in |
| in HVPG in response to zibotentan and | HVPG. |
| dapagliflozin in combination and | |
| dapagliflozin monotherapy versus placebo. | |
| To evaluate the effect of zibotentan and | Percentage and absolute change in spleen |
| dapagliflozin in combination and | stiffness from baseline to Week 6 and |
| dapagliflozin monotherapy versus placebo | Week 16. |
| on spleen | |
| stiffness as measured with | |
| VCTE/Fibroscan^{t}. | |
| To evaluate the effect of zibotentan and | Percentage and absolute change from |
| dapagliflozin in combination and | baseline in AST and ALT from baseline |
| dapagliflozin monotherapy versus placebo | to Week 6 and Week 16. |
| on liver health and portal hypertension | Absolute change from baseline in |
| biomarkers. | AST:ALT ratio, AST:platelet ratio, and |
| | FIB-4 from baseline to Week 6 and |
| | Week 16. |
| | Absolute change from baseline in |
| | MELD score from baseline to Week 6 |
| | and Week 16. |
| To evaluate the effect of zibotentan and | Composite of time to first event of: |
| dapagliflozin in combination and | Variceal haemorrhage. |
| dapagliflozin monotherapy versus placebo | Ascites (clinical grade 2 or 3)^{v}. |
| on proportion of participants experiencing a | Spontaneous bacterial peritonitis. |
| decompensation event defined as any of the | Hepatic encephalopathy (West Haven |
| following: variceal haemorrhage, clinically | grading)^{w}. |
| apparent ascites (grade 2 or 3), spontaneous | Acute kidney injury^{u}. |
| bacterial peritonitis, hepatic | MELD score > 15 on two consecutive |
| encephalopathy, acute kidney injury or | occasions (visits). |
| MELD score > 15 on two consecutive | Death. |
| occasions (visits) or death. | |
| To assess the plasma exposure of zibotentan | Plasma concentrations of zibotentan and |
| and dapagliflozin in the participant | dapagliflozin per treatment, visit and |
| population. | timepoint. |
| To determine the effect of zibotentan and | Change in eGFR from baseline to Week 1, |
| dapagliflozin in combination and | Week 6 and Week 16. |
| dapagliflozin monotherapy versus placebo | |
| on eGFR. | |
| To assess the effect of zibotentan and | Evaluation of changes in blood and urine |
| dapagliflozin in combination and | biomarkers across time course of the study. |
| dapagliflozin monotherapy versus placebo | |
| on plasma/serum potassium, sodium, | |
| creatinine, cystatin C, uric acid, plasma | |
| osmolality, BUN, BNP, fasting plasma | |
| glucose, fasting insulin, fasting | |
| beta-hydroxybutyrate, HbA1c, haematocrit, | |
| haemoglobin, ET-1, ELDP, CTproET-1, | |
| and copeptin levels, and on and urine | |
| sodium, potassium, urea, uric acid, glucose, | |
| and albumin creatinine ratios as well as | |
| urine osmolality. | |
| To collect and store plasma, serum and | Evaluation of changes in blood and urine |
| urine samples for potential exploratory | biomarkers across time course of the study. |
| research aimed at exploring biomarkers | |
| involved in PK, PD, safety and tolerability | |
| related to zibotentan and dapagliflozin in | |
| combination and dapagliflozin monotherapy | |
| versus placebo or related to liver diseases. | |
| Collect and store blood samples for genetic | Exploratory research into genes/genetic |
| research (according to each country's local | variation that may influence response to |
| and ethical procedures) (optional). | treatment. |

a. Home-based balance.
b. Office balance.
c. 40 mg furosemide = 1 mg bumetanide = 20 mg torsemide = 50 mg ethacrynic acid.
^{t}VCTE/Fibroscan assessment of spleen stiffness should only be done if technically feasible to complete an accurate spleen stiffness measurement and is up to the discretion of the investigator at the study centre.
^{u}AKI is defined as any of the following: increase in s-creatinine by 0.3 mg/dL (26.5 μmol/L) within 48 hours; or increase in s-creatinine to × 1.5 times baseline, which is known or presumed to have occurred within the prior 7 days (or using the last available value of outpatient s-creatinine within 3 months as the baseline value); or urine volume < 0.5 mL/kg/h for 6 hours.
^{v}Clinical grading of ascites: Grade 1; mild ascites, only detectable by imaging/ultrasound examination. Grade 2; moderate ascites, manifested by moderate symmetrical distension of abdomen. Grade 3; large or gross ascites that provokes marked abdominal distension.
^{w}West Haven criteria on HE: No HE. Minimal HE; psychometric or neuropsychological alterations of tests exploring psychomotor speed/executive functions or neurophysiological alterations without clinical evidence of mental change. Grade 1; trivial lack of awareness, euphoria or anxiety, shortened attention span, impairment of addition or subtraction, altered sleep rhythm. Grade 2; lethargy or apathy, disorientation for time, obvious personality change, inappropriate behaviour, dyspraxia, asterixis. Grade 3; somnolence to semi-stupor, responsive to stimuli, confused, gross disorientation, bizarre behaviour. Grade 4; coma.
Abbreviations: AE = adverse event; AESI = adverse event of special interest; AKI = acute kidney injury; ALT = alanine aminotransferase; AST = aspartate aminotransferase; BNP = B-type natriuretic peptide; BUN = blood urea nitrogen; CTproET-1 = C-terminal pro-endothelin-1; DAE = discontinuation due to adverse event; ECG = electrocardiogram; eGFR = estimated glomerular filtration rate; ELDP = endothelin-like domain peptide; ET-1 = endothelin-1; FIB-4 = fibrosis-4; GI = genital infection; HbA1c = haemoglobin A1c; HE = hepatic encephalopathy; HVPG = hepatic venous pressure gradient; MELD = model for end-stage liver disease; PD = pharmacodynamic; PK = pharmacokinetic; SAE = serious adverse event; UTI = urinary tract infection; VCTE = vibration-controlled transient elastography.

### REFERENCES

Bai et al 2021: Bai W, Al-Karaghouli M, Stach J, Sung S, Matheson G J, Abraldes J G. Test-Retest Reliability and Consistency of HVPG and Impact on Trial Design: A Study in 289 Patients from 20 Randomized Controlled Trials. Hepatology. 2021; 74:3301-15.
D'Amico et al, 2006: D'Amico G, García-Pagán J C, Luca A, Bosch J. Hepatic vein pressure gradient reduction and prevention of variceal bleeding in cirrhosis: a systematic review. Gastroenterology. 2006; 131(5): 1611 24.
Dapagliflozin Investigator's Brochure: Dapagliflozin Investigator's Brochure, current version.
García-Pagán et al, 2020: García-Pagán J C, Saffo S, Mandorfer M, Garcia-Tsao G. Where does TIPS fit in the management of patients with cirrhosis? JHEP Rep. 2020 May 23; 2(4):100122.
GBD 2017 Cirrhosis Collaborators, 2020: GBD 2017 Cirrhosis Collaborators. The global, regional, and national burden of cirrhosis by cause in 195 countries and territories, 1990-2017: a systematic analysis for the Global Burden of Disease Study 2017. Lancet Gastroenterol Hepatol. 2020 March; 5(3):245-266.
Gordon-Montalvo et al, 2020: Montalvo-Gordon I, Chi-Cervera L A, García-Tsao G. Sodium-Glucose Cotransporter 2 Inhibitors Ameliorate Ascites and Peripheral Edema in Patients with Cirrhosis and Diabetes. Hepatology 2020 November; 72(5):1880-1882. doi: 10.1002/hep.31270.
Groszmann and Wongcharatrawee, 2004: Groszmann R J and Wongcharatrawee S. The Hepatic Venous Pressure Gradient: Anything Worth Doing Should Be Done Right. Hepatology 2004: 39(2):280 90.
Gunarathne et al, 2020: Gunarathne L S, Rajapaksha H, Shackel N, Angus P W, Herath C B. Cirrhotic portal hypertension: From pathophysiology to novel therapeutics. World J Gastroenterol. 2020 Oct. 28; 26(40):6111-6140.
Hsiang et al, 2020: John Chen Hsiang, Vincent Wai-Sun Wong. SGLT2 Inhibitors in Liver Patients. Clin Gastroenterol Hepatol. 2020 September; 18(10):2168-2172.e2. doi: 10.1016/j.cgh.2020.05.021. Epub 2020 May 16.
Inker et al, 2021: Inker L A, Eneanya N D, Coresh J, Tighiouart H, Wang D, Sang Y, et al. New Creatinine and Cystatin C-Based Equations to Estimate GFR without Race. N Engl J Med. 2021; 385:1737 49.
Iwakiri, 2014: Iwakiri Y. Pathophysiology of portal hypertension. Clin Liver Dis. 2014 May; 18(2):281-91.
Kosiborod et al, 2021: Kosiborod M N, Esterline R, Furtado R H M, Oscarsson J, Gasparyan S B, Koch G G, et al. Dapagliflozin in patients with cardiometabolic risk factors hospitalised with COVID-19 (DARE-19): a randomised, double-blind, placebo-controlled, phase 3 trial. Lancet Diabetes Endocrinol. 2021 September; 9(9):586-594.
Levey et al, 2009: Levey A S, Stevens L A, Schmid C H, Zhang Y L, Castro A F, Feldman H I, et al. A new equation to estimate glomerular filtration rate. Ann Intern Med. 2009; 150:604-12.
Morris et al, 2005: Morris C D, Rose A, Curwen J, Hughes A M, Wilson D J, Webb D J. Specific inhibition of the endothelin A receptor with ZD4054: clinical and pre-clinical evidence. Br J Cancer. 2005 Jun. 20; 92(12):2148-52.
Nelson et al, 2012: Nelson J B, Fizazi K, Miller K, Higano C, Moul J W, Akaza H, et al. Phase III, randomized, placebo-controlled study of zibotentan (ZD4054) in patients with castration-resistant prostate cancer metastatic to bone. Cancer. 2012 Nov. 15; 118(22):5709-18.
Okamoto et al, 2016: Okamoto T, Koda M, Miyoshi K, Onoyama T, Kishina M, Matono T, et al. Antifibrotic effects of ambrisentan, an endothelin-A receptor antagonist, in a non-alcoholic steatohepatitis mouse model. World J Hepatol. 2016; 8(22):933-41.
Poordad, 2015: Poordad F F. Presentation and complications associated with cirrhosis of the liver. Curr Med Res Opin. 2015 May; 31(5):925-37.
Rockey and Chung, 1996: Rockey D C and Chung J J. Endothelin antagonism in experimental hepatic fibrosis. Implications for endothelin in the pathogenesis of wound healing. J Clin Invest. 1996; 98: 1381 88.
Romanelli et al, 2016: Romanelli R G, Stasi C. Recent advancements in diagnosis and therapy of liver cirrhosis. Curr Drug Targets. 2016; 17(15):1804-17.
Takahashi et al, 2021: Takahashi H, Kessoku T, Kawanaka M, Nonaka M, Hyogo H, Fujii H, et al. Ipragliflozin Improves the Hepatic Outcomes of Patients with Diabetes with NAFLD. Hepatol Commun. 2021 Jun. 17. doi: 10.1002/hep4.1696. Online ahead of print.
Tomkinson et al, 2011: Tomkinson H, Kemp J, Oliver S, Swaisland H, Taboada M, Morris T. Pharmacokinetics and tolerability of zibotentan (ZD4054) in subjects with hepatic or renal impairment: two open label comparative studies. BMC Clin Pharmacol, 2011; 11-3.
Treem et al, 2021: Treem W R, Palmer M, Lonjon-Domanec I, Seekins D, Dimick-Santos L, Avigan M I, et al. Consensus guidelines: Best practices for detection, assessment and management of suspected acute drug-induced liver injury during clinical trials in adults with chronic viral hepatitis and adults with cirrhosis secondary to Hepatitis B, C and non-alcoholic steatohepatitis. Drug Saf. 2021; 44(2):133-65.
Tsochatzis et al, 2014: Tsochatzis E A, Bosch J, Burroughs A K. Liver cirrhosis. Lancet. 2014; 383(9930):1749-61.
Zibotentan Investigator's Brochure: Zibotentan Investigator's Brochure, current version.
Zipprich et al, 2021: Zipprich A, Gittinger F, Winkler M, Dollinger M M, Ripoll C. Effect of ET-A blockade on portal pressure and hepatic arterial perfusion in patients with cirrhosis: A proof of concept study. Liver Int. 2021 March; 41(3):554-561.

1. A method of treating liver cirrhosis in a subject in need thereof, comprising administering to the subject a composition comprising a therapeutically effective amount of zibotentan and dapagliflozin.

2. The method of claim 1, wherein the subject has features of portal hypertension.

3. The method of claim 1, wherein the subject has a hepatic venous pressure gradient (HVPG) greater than 5 mmHg.

4. The method of claim 1, wherein the subject has an HVPG greater than 7.5 mmHg.

5. The method of claim 4, wherein the subject has an HVPG greater than 10 mmHg.

6-16. (canceled)

17. The method of claim 1, wherein the composition comprises about 0.5 mg to about 20 mg zibotentan.

18-23. (canceled)

24. The method of claim 1, wherein the composition comprises about 1 mg to about 10 mg dapagliflozin.

26-73. (canceled)

74. A method of reducing the risk of variceal haemorrhage and/or death, or preventing and/or delaying progression of variceal haemorrhage and/or death, in a subject in need thereof, comprising administering to the subject a composition comprising a therapeutically effective amount of zibotentan and dapagliflozin.

75. (canceled)

76. (canceled)

77. A method of reducing the risk of cirrhosis decompensation or preventing and/or delaying progression of cirrhosis decompensation, in a subject in need thereof, comprising administering to the subject a composition comprising a therapeutically effective amount of zibotentan and dapagliflozin.

78. (canceled)

79. (canceled)

80. The method of claim 74, wherein the subject has features of portal hypertension.

81. The method of claim 77, wherein the subject has features of portal hypertension.

82. The method of claim 74, wherein the subject has a hepatic venous pressure gradient (HVPG) greater than 5 mmHg.

83. The method of claim 77, wherein the subject has a hepatic venous pressure gradient (HVPG) greater than 5 mmHg.

84. The method of claim 74, wherein the composition comprises about 0.5 mg to about 20 mg zibotentan.

85. The method of claim 77, wherein the composition comprises about 0.5 mg to about 20 mg zibotentan.

86. The method of claim 74, wherein the composition comprises about 1 mg to about 10 mg dapagliflozin.

87. The method of claim 77, wherein the composition comprises about 1 mg to about 10 mg dapagliflozin.
